(19) United States
(12) Patent Application Publication
(10) Pub. No.: US 2008/0031874 A1
(43) Pub. Date: Feb. 7, 2008

(54) Treatment of endotoxemia using endotoxin neutralizing agents

(76) Inventor: Martin E. Sanders, Hillsborough, CA (US)

Correspondence Address: MOORE PATENTS, 794 LOS ROBLES AVENUE, PALO ALTO, CA, 94306-3159, US

(21) Appl. No.: 11/703,538

(22) Filed: Feb. 6, 2007

Related U.S. Application Data
(60) Provisional application No. 60/765,996, filed on Feb. 6, 2006.

(51) Int. Cl. A61K 39/40; A61K 39/00; A61P 43/00; C12Q 1/00

(52) U.S. Cl. 424/133.1; 424/150.1; 424/164.1; 435/4

(57) ABSTRACT

Methods and compositions for selecting a patient suffering from endotoxemia for treatment with an endotoxin neutralizing agent are disclosed comprising: (a) determining the level of endotoxin in the patient's blood; and (b) comparing the endotoxin level in the patient's blood to a predetermined threshold endotoxin level to determine if the patient has elevated endotoxin levels. The methods can further comprise treating patients identified as having elevated levels of endotoxin with an endotoxin neutralizing therapy. The methods provide increased safety and a reduction in risk for critically ill patients.

### CROSS-REFERENCE TO RELATED APPLICATIONS

This application claims priority from U.S. Provisional Patent Application Ser. No. 60/765,996, filed Feb. 6, 2006, incorporated by reference herein in its entirety.

### FIELD OF THE INVENTION

This invention relates generally to diagnosis and selection of patients for treatment and methods for treating conditions associated with endotoxemia and the like.

### BACKGROUND OF THE INVENTION

Medical researchers have long sought effective therapeutic approaches and compositions for treating patients suffering from gram negative bacteremia, sepsis and septic shock, and related conditions. It was reported that immunization of animals against gram negative bacteria provided protection from the effects of endotoxin, lethal shock and infection. For example, Nys et al. reported that anti-LPS antibodies attenuated the effects of gram negative sepsis but were not sufficient to prevent endotoxemia in rodents challenged with gram negative bacterial infection. Nys, M., et al., (1999) Med. Microbiol. Immunol. 188(2), 55-64. In humans, antiserum obtained from human volunteers immunized against the core glycolipid region of lipopolysaccharide (LPS), J5, was reported to reverse established shock and reduce deaths from gram negative bacteremia. Ziegler, E. J., et al. (1982) New Engl. J. Med. 307, 1225; Braude, A. I., et al., (1981) Am. J. Med. 70(2), 463-466. The prophylactic effect of the anti-J5 antiserum was tested in abdominal surgery patients and was reported to prevent the serious consequences of gram-negative infections even though it did not decrease the incidence of infection. Baumgartner, J. D., et al., (1985) The Lancet 2(8446), 59-63.

As a result of these favorable reports, monoclonal antibodies were developed having specific binding for J5 and tested in clinical trials in human patients. HA-1A, a human monoclonal antibody developed by Centocor, and E5, a murine monoclonal antibody developed by Xoma, were tested in human patients with mixed results. Initial studies indicated positive results. However, larger trials failed to demonstrate significant benefit, and in fact, appeared to indicate that some patients were put at higher risk for mortality.

One large clinical trial showed no overall benefit to patients with sepsis who did not prove to have gram-negative bacteremia from administering HA-1A, but significant improvement in the survival rate was reported in a subgroup of patients with gram-negative bacteremia and shock. Ziegler, E. J., et al. (1991) New Engl. J. Med. 324, 429-436. The authors reported that the effect was specific for patients with sepsis and gram-negative bacteremia, and that there was no significant difference in mortality for patients with nonbacteremic gram-negative infection, patients with gram-positive infection, patients with fungal infection or patients with no infection identified. They suggested that the results of the trial should only be extended to patients who are septic and presumed gram-negative bacteremic.

However, a second trial did not confirm the improved survival rate in patients with gram-negative bacteremia and was discontinued at the first interim analysis due to a survival disadvantage among patients without gram-negative bacteremia (42% mortality among patients who received HA-1A relative to 38% mortality for placebo). McCloskey, R. V., et al. (1994) Ann. Intern. Med. 121, 1-5. The authors speculated that the conclusions of the previous trial of HA-1A may have been incorrect and HA-1A may not be effective in patients with sepsis and gram-negative bacteremia. They concluded that septic shock should not be used as an indication for treatment with HA-1A, and suggested that other criteria for patient selection are needed to identify patients who are severely ill and dying from endotoxemia. However, no other criteria were suggested.

Fulminant meningococcemia was suggested as a model to test the efficacy of HA-1A. However, in a subsequent trial of HA-1A in children suffering from meningococcal septic shock, no statistically significant benefit was demonstrated in terms of mortality. Derkx, B., et al. (1999) Clin. Infect. Diseases 28, 770-777. In this article, the authors stated that no single endotoxin antibody therapeutic strategy had been shown to improve the clinical outcome for patients with sepsis syndrome or septic shock. They further speculated that patients dying from endotoxemia are most likely to benefit from anti-endotoxin therapy, but stated that it has been impossible to identify patients with gram-negative bacteremia and/or endotoxemia at an early stage. They further suggest that future clinical trials should identify a restricted and homogenous patient population that might benefit from therapy with the anti-endotoxin antibody.

In a canine model of gram negative septic shock, it was reported that animals receiving HA-1A did not show altered levels of bacteremia or endotoxemia and that survival was decreased. Quezado, Z. M. N., et al. (1993) J. Amer. Med. Assoc. 269, 2221-2227. The authors concluded that the conditions under which HA-1A has beneficial, neutral or deleterious effects needs to be established before widespread use of the antibody could be permitted.

Greenman, et al. reported that the mouse monoclonal anti-endotoxin antibody E5 did not demonstrate increased survival among all patients tested, and showed benefit only to patients with gram negative sepsis who were not in shock at study entry (Greenman, R. L., et al. (1991) J. Amer. Med. Assoc. 266, 1097-1102). In a commentary about these anti-endotoxin antibody studies, Bone stated that studies testing the usefulness of the mouse and human monoclonal anti-endotoxin antibodies E5 and HA-1A, respectively, reported that both antibodies can reduce mortality and increase multiorgan failure reversal in some patients with gram negative sepsis, but that the antibodies should not be used indiscriminately. Bone, R. C. (1991) J. Amer. Med. Assoc. 266, 1125-1126, emphasis in original). Bone stated that, first and foremost, no patient should be given either antibody unless gram-negative sepsis was strongly suspected, and that patients should not receive the antibodies unless their clinical condition matches the definition of sepsis used in the studies. Bone cautioned against treating any patient whose condition is dissimilar to the condition of patients tested, for example, neutropenic patients. Bone further suggests that if culture results or endotoxin assays were available, the decision to dose with anti-endotoxin antibodies might be easier, however he does not explain how the decision should be made, and cautions against the unrestrained use of this therapy in the large population of patients who are unlikely to benefit. Another commentator characterizes Bone as suggesting that the presence of endotoxemia may identify a population of patients who could benefit from the administration of antibodies against endotoxin. Balk, R. A. (2002) Crit. Care 6, 289-290. However, there is no teaching that would elucidate how such an assay could be implemented or interpreted for clinical benefit of patients other than as a confirmation of gram negative sepsis.

More recently, U.S. Patent Application Publication No. 200600512821 to Rossignol describes a method of determining whether a patient could benefit from treatment with a toll-like receptor 4 (TLR4) antagonist comprising contacting a sample from the patient that comprises infected tissue or fluid comprising white blood cells with an antibody that binds specifically to an indicator of gram negative bacterial infection, and detecting the level of oxidants produced by white blood cells in the sample in the presence of the antibody as a measure of the level of the indicator in the sample, wherein detection of an increased level of the indicator relative to a negative control indicates that said patient could benefit from treatment with a TLR4 antagonist. However, this application describes treatment with TLR4 antagonists, and does not describe how to determine if a patient could benefit from administration of TLR4 antagonists beyond any positive result on assay for any indicator of gram negative bacterial infection.

Further, endotoxemia was reported to be a weak prognostic indicator, and appeared to have most prognostic significance when coinciding with gram negative bacteremia. Hurley, J. C. (2003) J. Endotoxin Res. 9(5), 271-279. Early detection of endotoxemia was reported to be associated with gram negative bacterial infection only in cases of bacteremic infection, but early endotoxemia did not correlate with organ dysfunction or mortality in patients with severe sepsis or septic shock. Venet, C., et al., (2000) Intensive Care Med. 26(5), 538-544. Another author reported that endotoxemia is present in the blood of only about 30% of patients with bacteremia, and concluded that endotoxemia does not predict gram negative bacteremia, gram negative infection, or survival from sepsis. Cohen, J. (2000) Intensive Care. Med. 26, S51-6. Cohen further opined that there is no place for routine endotoxin testing in clinical practice because the positive predictive value of the test for gram negative bacteremia is insufficiently high to be of clinical use.

Complicating matters is the fact that patients can have gram negative bacteremia without endotoxemia, endotoxemia without gram negative bacteremia, endotoxemia with gram positive bacteremia, and endotoxemia with leaky bowel syndrome, without any bacteremia in evidence at all. Thus, confusion exists as to what conditions are appropriate for treatment of patients with endotoxin neutralizing agents for prevention and/or treatment of septic shock and associated multiorgan failures, morbidity and mortality. As treatment of inappropriate patient populations appears to be associated with toxicity and increased mortality, therefore there is an urgent need to identify patients at risk from endotoxemia who can actually benefit from treatment with endotoxin neutralizing therapies. Heretofore, such identification has eluded clinicians and researchers.

In summary, the literature provides conflicting results regarding clinical efficacy and indications for the use of anti-endotoxin therapeutic agents to treat humans suffering from or at risk of developing septic shock, and even urges caution regarding use of anti-endotoxin therapeutic agents because of adverse events and excess mortality in some cases. The appropriate timing and dosing of anti-endotoxin therapies has not been established, and anti-endotoxin therapeutics are not approved for marketing for use in humans. Thus, there remains a critical unmet need to treat patients who develop endotoxemia and associated pathological conditions.

### SUMMARY OF THE INVENTION

Accordingly, it is therefore an object of the present invention to provide methods and compositions for treating endotoxemia and conditions characterized by elevated levels of endotoxin with enhanced safety.

It is an additional object of the invention to provide methods and compositions for treating endotoxemia to substantially reduce the risk of a patient developing sepsis, septic shock, SIRS or MODS; to provide reduced morbidity and mortality; and to substantially reduce the risk of toxic reactions and/or untoward side effects of the endotoxin neutralizing agent when administered to an inappropriate patient population.

Accordingly, in one embodiment, methods for selecting a patient suffering from endotoxemia for treatment with an endotoxin neutralizing agent are provided comprising: (a) determining the level of endotoxin in the patient's blood; and (b) comparing the endotoxin level in the patient's blood to a predetermined threshold endotoxin level to determine if the patient has elevated endotoxin levels. The methods can further comprise administering a therapeutically effective amount of an endotoxin neutralizing agent to patients identified as having elevated levels of endotoxin. In one embodiment, the elevated level of endotoxin is from about 5 pg/ml to at least about 100 pg/ml. In another embodiment, the elevated level of endotoxin is from about 5 pg/ml to about 60 pg/ml. In additional embodiment, the threshold endotoxin level is from about 5 to about 20 pg/ml. Preferably, the patient is a mammal and more preferably, the patient is a human.

In one embodiment, the endotoxin neutralizing agent can be a monoclonal or polyclonal antibody that binds endotoxin. Preferably, the monoclonal antibody that binds endotoxin is HA-1A, mAb 216, or E5, or a fragment, a fusion protein, a chimera, recombinant versions thereof, or combinations thereof. In a preferred embodiment, the monoclonal antibody that binds endotoxin can be a VH4-34 antibody. In further embodiments, the monoclonal antibody is an IgM or an IgG, or can be an IgG, or an IgG₃, or a fragment, a fusion protein, a chimera, or combinations thereof. The polyclonal antibody can be, for example, Pentaglobin®, or purified antibodies obtained from serum or recombinant methods In additional embodiments, the endotoxin neutralizing agent is a LPS binding protein, a bactericidal permeability increasing protein, or the like.

In particular embodiments, the endotoxemia is associated with gram negative bacteremia. In additional embodiments, the endotoxemia is associated with gram positive bacteremia or fungemia. In certain other embodiments, the endotoxemia is present without documentable bacteremia or fungemia. In yet other embodiments, the endotoxemia is associated with infection with meningococcus. In additional embodiments, the endotoxemia is associated with a biowarfare agent, such as Yersinia pestis, Franciella tularensis, Shigella sp., Salmonella sp., or other gram negative bacteria. In other embodiments, the endotoxemia is associated with liver disease, pancreatitis, neutropenia or other immune suppression. In additional embodiments, the endotoxemia is associated with bowel edema/leaky gut due to severe systemic illness such as infection, trauma, ischemia, chemotherapy, radiation therapy, post-surgical state, or multiorgan failure, where typically the multiorgan failure is selected from liver, lung, renal or cardiac failure.

The methods can further comprise administering an additional active agent. Preferably, the additional active agent is selected from an antibiotic, additional endotoxin neutralizing agents, such as a TLR-4 receptor antagonist, or a cytokine inhibitor, an anti-inflammatory agent, or an anticoagulant, or combinations thereof. Suitable TLR-4 receptor antagonists include for example, E5564 or TAK-242. Typical anti-inflammatory agents include nonsteroidal anti-inflammatory agents selected from salicylic acid derivatives, aryl propionic acids, heteroaryl acetic acids, indene acetic acids, selective COX-2 inhibitors, alkanones, oxicams, or anthranilic acids. Typical anticoagulants can include an activated protein C or heparin. Typical anti-inflammatory agents include anti-inflammatory steroids such as prednisone, prednisolone, methylprednisolone, triamcinolone or dexamethasone. Generally, the cytokine inhibitor is an inhibitor of IL-6, an IL-1, or a TNF.

In another aspect, methods are provided for treating a patient suffering from endotoxemia comprising: (a) determining the level of endotoxin in the patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin. Preferably, the patient is mammal, and more preferably, the patient is a human patient. The methods can further comprise administering an additional active agent, preferably selected from an additional endotoxin neutralizing agent, such as a TLR-4 receptor antagonist or an anti-endotoxin antibody, or a cytokine inhibitor, a nonsteroidal anti-inflammatory agent, a steroidal anti-inflammatory agent, or an anticoagulant, or combinations thereof. Typical TLR-4 receptor antagonists include E5564 or TAK-242. Anti-inflammatory agents include the nonsteroidal anti-inflammatory agents such as salicylic acid derivatives, aryl propionic acids, heteroaryl acetic acids, indene acetic acids, selective COX-2 inhibitors, alkanones, oxicams, or anthranilic acids, as well as anti-inflammatory steroids such as prednisone, prednisolone, methylprednisolone, triamcinolone, or dexamethasone. Anticoagulants can include an activated protein C or heparin.

Preferably, the elevated level of endotoxin is from about 5 pg/mL to at least about 100 pg/mL, and more preferably, from about 5 pg/mL to about 60 pg/mL. The threshold endotoxin level is preferably from about 5 to about 20 pg/mL.

The endotoxin neutralizing agent can be an anti-endotoxin antibody, such as a monoclonal or polyclonal antibody, a LPS binding protein, a bactericidal permeability increasing protein, or any other agent capable of functionally neutralizing endotoxin. In a preferred embodiment, the endotoxin neutralizing agent is a monoclonal antibody that binds endotoxin, for example, HA-1A, mAb 216, or E5, or fragments, fusion proteins, chimeras, or combinations thereof, or recombinant versions thereof. In a particular embodiment, the monoclonal antibody that binds endotoxin is a VH4-34 antibody. The monoclonal antibody can be an IgM or an IgG, preferably an IgG₁ or an IgG₃. or a fragment, a fusion protein, a chimera, recombinant versions thereof, or combinations thereof. In an additional embodiment, the endotoxin neutralizing agent can be a LPS binding protein or a bactericidal permeability increasing protein.

In particular embodiments, the endotoxemia can be associated with gram negative bacteremia. In additional embodiments, the endotoxemia can be associated with gram positive bacteremia or fungemia. In yet other embodiment, the endotoxemia is present without documentable bacteremia or fungemia. In certain embodiments, the endotoxemia is associated with infection with meningococcus. In yet other embodiments, the endotoxemia is associated with infection with a biowarfare agent, such as Yersinia pestis, Franciella tularensis, Shigella sp., Salmonella sp., or other gram negative bacteria. In alternative embodiments, the endotoxemia can be associated with neutropenia or other immune suppression. In yet other embodiments, the endotoxemia is associated with liver disease or pancreatitis. In still other embodiments, the endotoxemia is associated with bowel edema/leaky gut due to severe systemic illness such as infection, trauma, ischemia, chemotherapy, radiation therapy, post-surgical state, or multiorgan dysfunction syndrome (MODS), for example, where the multiorgan dysfunction syndrome is due to liver, renal, cardiac or lung failure. In additional embodiments, the endotoxemia is associated with peritonitis, neutropenia, urosepsis, severe liver injury, severe pancreatitis, leaky bowel syndrome, or meningococcemia.

In another aspect, methods are provided for controlling endotoxemia in a patient comprising: (a) determining the level of endotoxin in the patient's blood; (b) comparing the endotoxin level in the patient's blood to a predetermined threshold endotoxin level to determine if the patient has elevated endotoxin levels; and (c) treating patients identified as having elevated levels of endotoxin with a means for controlling endotoxemia. Preferably, the means for controlling endotoxemia comprises the administration of an endotoxin neutralizing agent or plasmapheresis to reduce the level of endotoxin in the patient's blood, or combinations thereof. Preferably, the level of endotoxin in the patient's blood is reduced below 100 pg/ml, more preferably to below about 5-20 pg/ml and most preferably to below the threshold of detection. The endotoxin neutralizing agent can be administered as a bolus or continuously over time. Preferably, the endotoxin level in the patient is maintained at or below the threshold level for endotoxemia. More preferably, the endotoxin level is reduced to undetectable levels.

In another aspect, methods are provided for preventing septic shock, SIRS, MODS or mortality in a patient comprising: (a) determining the level of endotoxin in the patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin.

In an additional aspect, methods are provided for reducing patient mortality, comprising: (a) determining the level of endotoxin in a patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin.

In yet another aspect, methods are provided for reducing hospital and/or intensive care unit duration comprising: (a) determining the level of endotoxin in a patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin. Preferably, the reduced hospital or intensive care unit duration is at least 0.5 days, more preferably one or more days, and most preferably at least two days reduced duration.

In other aspects, methods are provided for reducing the incidence of morbidities in a patient, comprising: (a) determining the level of endotoxin in the patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin. Typical morbidities include ARDS, SIRS, hepatic failure, renal failure, cardiac failure and MODS.

In additional aspects, methods are provided for monitoring the therapeutic efficacy of a treatment for endotoxemia in a patient in need thereof, comprising: (a) determining the level of endotoxin in the patient's blood; (b) performing a treatment for endotoxemia; (c) determining the level of endotoxin in the blood of a patient at a time after the treatment for endotoxemia; (d) comparing the endotoxin level in the patient's blood to a predetermined threshold endotoxin level or the level of endotoxin prior to treatment to determine whether the endotoxin level in the patient's blood has decreased due to the treatment for endotoxemia or whether the endotoxin level remains elevated. The methods can further comprise providing additional treatments for endotoxemia to patients identified as having elevated levels of endotoxin, for example, plasmapheresis or the administration of an endotoxin neutralizing agent, or combinations thereof. The treatments for endotoxemia can further comprise administering an additional active agent such as, but not limited to, an antibiotic, a TLR-4 receptor antagonist, a cytokine inhibitor, an anti-inflammatory agent, or an anticoagulant, or combinations thereof.

In an additional aspect, methods are provided for monitoring the therapeutic efficacy of a treatment for endotoxemia in a patient in need thereof, comprising: (a) determining the amounts of endotoxin and endotoxin neutralizing agent in the patient's blood; and (b) comparing the amount of endotoxin neutralizing agent to the amount of endotoxin in the patient's blood to determine if the patient has sufficient endotoxin neutralizing agent to functionally neutralize the endotoxin. The methods can further comprise (c) administering an endotoxin neutralizing agent to patients identified as having an insufficient amount of endotoxin neutralizing agent to functionally neutralize the endotoxin in the patient's blood, and the endotoxin neutralizing agent administered in step (c) can be the same or different from the endotoxin neutralizing agent present in the patient's blood. Preferred endotoxin neutralizing agents are selected from a LPS-binding protein, anti-endotoxin antibody, or bactericidal permeability inducing protein. In a particular embodiment, the anti-endotoxin antibody is an endogenous anti-endotoxin antibody or an exogenous anti-endotoxin antibody. In another embodiment, the anti-endotoxin antibody can be a VH4-34 antibody. In a preferred embodiment, the exogenous anti-endotoxin antibody is selected from HA-1A, E5, Mab 216, recombinant versions thereof, fragments, fusion proteins, chimeras, or combinations thereof.

In yet other aspects, methods are provided for monitoring the therapeutic efficacy of a treatment for endotoxemia in a patient in need thereof, comprising: (a) determining the amount of endotoxin neutralizing agent in the patient's blood; and (b) comparing the amount of endotoxin neutralizing agent in the patient's blood to the amount of endotoxin neutralizing agent that would be sufficient to functionally neutralize an endotoxin concentration of from about 5 to about 1000 pg/ml in the patient's blood to determine if the patient has sufficient endotoxin neutralizing agent in their blood to functionally neutralize an endotoxin concentration of from about 5 to about 1000 pg/ml. The methods can further comprise (c) administering an endotoxin neutralizing agent to patients identified as having an amount of endotoxin neutralizing agent in their blood that is not sufficient to functionally neutralize an endotoxin concentration of from about 5 to about 1000 pg/ml.

In another aspect, methods are provided for treating a patient suffering from endotoxemia, comprising: (a) determining the level of endotoxin in the patient's blood; (b) comparing the endotoxin level in the patient's blood to a predetermined threshold endotoxin level in the patient's blood to determine if the patient has elevated endotoxin levels; and (c) performing plasmapheresis on the patient's blood using a plasmapheresis system comprising an endotoxin binding agent for removing endotoxin from the patient's blood. In a preferred embodiment, the endotoxin binding agent is a monoclonal or polyclonal antibody that binds endotoxin. Preferred monoclonal antibodies that bind endotoxin include HA-1A, mAb 216, or E5, or a fragment, a fusion protein, a chimera, recombinant versions thereof, or combinations thereof. In a particular embodiment, the monoclonal antibody that binds endotoxin is a VH4-34 antibody. The monoclonal antibody or polyclonal antibodies can be an IgM or an IgG, or fragments, fusion proteins, chimeras, or combinations thereof. In an additional embodiment, the endotoxin binding agent is a LPS binding protein, a polymyxin (e.g., polymyxin B), bactericidal permeability increasing protein or any other endotoxin binding agent having a binding affinity for endotoxin of at least 10⁶ M⁻¹.

In yet another aspect, methods are provided for treating a patient suffering from a condition characterized by elevated levels of plasma endotoxin, comprising: (a) determining the level of endotoxin in the patient's blood; (b) comparing the endotoxin level in the patient's blood to a predetermined threshold endotoxin level in the patient's blood to determine if the patient has elevated endotoxin levels; and (c) administering an endotoxin neutralizing agent to patients identified as having elevated levels of endotoxin, said method providing a reduced risk of sepsis, septic shock, SIRS, MODS or mortality, and having improved safety relative to a method for administering an endotoxin neutralizing agent in the absence of elevated levels of plasma endotoxin. The methods provide an enhanced safety margin for administering the endotoxin neutralizing agent, reducing the risk of adverse events and mortality to the patient, because the endotoxin neutralizing agent is administered to the patient based on an objective measurement of a need for additional endotoxin neutralizing agent, and not on severity of illness or documented infection or other signs.

In yet other aspects, methods are provided for preventing septic shock, SIRS, MODS or mortality in a patient comprising: (a) determining the level of calcitonin precursors in the patient's blood; (b) comparing the level of calcitonin precursors in the patient's blood to a predetermined threshold level of calcitonin precursors in the patient's blood to determine if the patient has elevated calcitonin precursor levels; and if elevated, then measuring endotoxin levels in the blood of patients having elevated calcitonin precursors; and (c) administering an endotoxin neutralizing agent to patients identified as having elevated levels of calcitonin precursors and endotoxin.

In other aspects, methods are provided for reducing the risk of septic shock, SIRS, MODS or mortality in a patient suffering from pancreatitis or a condition characterized by leaky bowel syndrome or failure of gut barrier function, comprising: (a) determining the level of calcitonin precursors in the patient's blood; (b) comparing the level of calcitonin precursors in the patient's blood to a predetermined threshold calcitonin precursors in the patient's blood to determine if the patient has elevated calcitonin precursor levels; and if elevated, then measuring endotoxin levels in the blood of patients having elevated calcitonin precursors; and (c) administering an endotoxin neutralizing agent to patients identified as having elevated levels of calcitonin precursors and endotoxin.

In additional aspects, methods are provided for treating a patient at risk for developing septic shock, SIRS, MODS or mortality, comprising: (a) determining the level of endotoxin in the patient's blood; (b) comparing the endotoxin level in the patient's blood to a predetermined threshold endotoxin level in the patient's blood to determine if the patient has elevated endotoxin levels; and (c) administering a polyclonal antibody composition having binding specificity for at least one endotoxin epitope to patients identified as having elevated levels of endotoxin. Preferably, the polyclonal antibody composition has binding specificity for more than one endotoxin epitope.

In another aspect, pharmaceutical compositions are provided for treating a patient suffering from endotoxemia, comprising an effective amount of at least one endotoxin neutralizing agent and a pharmaceutically acceptable carrier. In a particular embodiment, the composition comprises at least two endotoxin neutralizing agents. In another embodiment, the endotoxin neutralizing agent is at least one monoclonal antibody that binds endotoxin, such as, but not limited to, HA-1A, mAb 216, or E5, or fragments, fusion proteins, chimeras, or recombinant versions thereof, or combinations thereof. In a particular embodiment, the monoclonal antibody that binds endotoxin is a VH4-34 antibody. The monoclonal antibody can be an IgM or an IgG, preferably an IgG₁ or an IgG₃, or a fragment, a fusion protein, a chimera, recombinant versions thereof, or combinations thereof. In another embodiment, the at least one endotoxin neutralizing agent is a LPS binding protein or a bactericidal permeability increasing protein or a TLR-4 receptor antagonist. In certain particular embodiments, the composition comprises at least two of the following: a monoclonal antibody that binds endotoxin, a LPS binding protein, a bactericidal permeability increasing protein, or combinations thereof. The composition can further comprise an additional active agent, such as, but not limited to, an antibiotic, a TLR-4 receptor antagonist, a cytokine inhibitor, an anti-inflammatory agent, or an anticoagulant, or combinations thereof. In a preferred embodiment, the composition comprises at least one endotoxin neutralizing agent and a TLR-4 receptor antagonist. In an additional embodiment, a composition is provided for treating a patient suffering from endotoxemia, comprising a polyclonal antibody composition having binding specificity for at least one endotoxin epitope. The polyclonal antibody composition can have binding specificity for more than one endotoxin epitope.

In additional aspects, kits are provided for selecting a patient suffering from endotoxemia for treatment with an endotoxin neutralizing agent comprising: (a) means for determining the level of endotoxin in the patient's blood; and (b) instructions for determining whether the patient has elevated levels of endotoxin in their blood; and optionally, (c) at least one dose of a therapeutic composition comprising an endotoxin neutralizing agent.

In a further aspect, kits are provided for treating a patient suffering from endotoxemia comprising: (a) means for determining the level of endotoxin in the patient's blood; (b) instructions for determining whether the patient has elevated levels of endotoxin in their blood; and (c) at least one dose of a therapeutic composition comprising an endotoxin neutralizing agent.

In an additional aspect, a kit is provided for monitoring the therapeutic efficacy of a treatment for endotoxemia in a patient in need thereof, comprising: (a) means for determining the level of endotoxin in the patient's blood before and after performing a treatment for endotoxemia; (b) instructions for determining whether the patient has elevated levels of endotoxin in their blood; and optionally, (c) at least one dose of a therapeutic composition comprising an endotoxin neutralizing agent.

In a further embodiment, kits are provided for monitoring the therapeutic efficacy of a treatment for endotoxemia in a patient in need thereof, comprising: (a) means for determining the amounts of endotoxin and endotoxin neutralizing agent in the patient's blood; and (b) instructions for determining whether the patient has elevated levels of endotoxin in their blood and whether the patient has sufficient amount of endotoxin neutralizing agent to functionally neutralize the amount of endotoxin in their blood or an amount of endotoxin of between about 5 and at least about 1000 pg/ml.

In another aspect, kits are provided for preventing septic shock, SIRS, MODS or mortality in a patient comprising: (a) means for determining the level of calcitonin precursors and endotoxin levels in the patient's blood; (b) instructions for determining whether the patient has elevated calcitonin precursor and endotoxin levels; and optionally, (c) at least one dose of an endotoxin neutralizing agent.

In another aspect, kits are provided kit for reducing the risk of septic shock, SIRS, MODS or mortality in a patient suffering from pancreatitis or a condition characterized by leaky bowel syndrome or failure of gut barrier function, comprising: (a) means for determining the level of calcitonin precursors and endotoxin in the patient's blood; (b) instructions for determining if the patient has elevated calcitonin precursors and endotoxin levels; and optionally, (c) at least one dose of an endotoxin neutralizing agent.

In another aspect, the use of an endotoxin neutralizing agent in the manufacture of a medicament for the treatment of endotoxemia is provided.

Additional objects, advantages and novel features of the invention will be set forth in part in the description which follows, and in part will become apparent to those skilled in the art upon examination of the following, or may be learned by practice of the invention.

### DETAILED DESCRIPTION OF THE INVENTION

I. Definitions and Overview

Before the present invention is described in detail, it is to be understood that unless otherwise indicated this invention is not limited to specific antibodies, antibody fragments, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “an endotoxin neutralizing agent” can include one or more endotoxin neutralizing agents; reference to “an active agent” includes two or more pharmaceutically active agents, and so forth.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The term “antibody” is used in the broadest sense and specifically covers intact natural antibodies, antibodies produced using recombinant methods, monoclonal antibodies, polyclonal antibodies, multispecific antibodies (e.g. bispecific antibodies) formed from at least two intact antibodies, synthetic antibodies such as tetravalent antibodies, “evibodies” which are antibody compositions derived from CTLA-4 and related proteins, as described in U.S. Pat. No. 7,166,697 to Galanis, and antibody fragments, so long as they exhibit the desired biological activity. Human antibodies include antibodies made in nonhuman species. The term antibody also encompasses Ig molecules formed only from heavy chains, such as those obtained from Camelids, and described in U.S. Pat. Nos. 6,765,087 and 6,015,695 to Casterman, for example. The term antibody also encompasses fusion or chemical coupling (i.e., conjugation) of antibodies with labels and detection agents.

“Antibody fragments” comprise a portion of an intact antibody, preferably the antigen binding or variable region of the intact antibody. Examples of antibody fragments include Fab, Fab′, F(ab′)₂, and Fv fragments; diabodies, triabodies, tetrabodies; linear antibodies (Zapata, et al. (1995) Protein Eng. 8(10),1057-1062) single-chain antibody molecules; and multispecific antibodies formed from antibody fragments.

The term “blood” refers to all components of blood, including whole blood, serum, plasma, cell fractions, and can refer to bodily fluids other than blood that can be utilized instead of blood for determining elevated levels of endotoxin, endotoxin neutralizing agents or calcitonin precursors or proinflammatory cytokines. For example, a sample of a patient's lymph or ascites may be obtained and tested for endotoxin to determine if the patient is suffering from endotoxemia.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256, 495 (1975), or may be made by recombinant DNA methods (see, e.g., U.S. Pat. No. 4,816,567). The “monoclonal antibodies” can also be isolated from phage antibody libraries using the techniques described in Clackson et al. (1991) Nature 352, 624-628 and Marks et al., (1991) J. Mol. Biol. 222, 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., (1984) Proc. Natl. Acad. Sci. USA, 81, 6851-6855).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat or rabbit having the desired specificity, affinity, and capacity. In some instances, framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDRs correspond to those of a non-human immunoglobulin and all or substantially all of the FRs are those of a human immunoglobulin sequence. The humanized antibody optimally also will comprise at least a portion of an immunoglobulin constant region (Fc), typically that of a human immunoglobulin. For further details, see Jones et al., (1986) Nature 321, 522-525; Reichmann et al., (1988) Nature 332, 323-329; and Presta (1992) Curr. Op. Struct. Biol. 2, 593-596. The humanized antibody includes a PRIMATIZED™ antibody wherein the antigen-binding region of the antibody is derived from an antibody produced by immunizing macaque monkeys with the antigen of interest.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH—VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90, 6444-6448.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “conjugate” refers to coupling of active agents, which can be covalent or noncovalently associated. Typically, endotoxin neutralizing agents can be conjugated to enzymes, dyes, or labels, for example, in order to facilitate their use in kits or assays for monitoring therapeutic efficacy.

The term “9G4” refers to the rat monoclonal antibody that has been shown to recognize VH4-34 Ab (Stevenson, et al. (1986) Blood 68, 430). The VH4-34 epitope identified by mAb 9G4 is conformation restricted and dependent on a unique sequence near amino acids 23-25 in the framework 1 region (“FR1”) of the variable heavy chain. The VH4-34 gene has low incidence of mutation, allowing the reliable detection of VH4-34 antibodies using 9G4 by standard immunoassay methods.

The term “VH4-34 antibody” refers to one of the 53 identified human functional antibody germline antibodies, encoded by germline genes (VH4.21). Cook, G. P., et al., (1994) Nat. Genet. 7, 162-168. The gene for VH4-34 antibodies is present in all haplotypes and no sequence variation has been reported in germline DNA isolated from unrelated individuals. Weng, N. P., et al., (1992) Eur. J. Immunol. 22, 1075-1082; van der Maarel, S., et al., (1993) J. Immunol. 150, 2858-2868. Antibodies encoded by the VH4-34 gene have been shown to possess unique properties. All mAbs directed against the “I” or “i” antigens of red blood cells (RBCs) are encoded by the VH4-34 gene, are generally of the IgM class, and are classically described as cold agglutinins (CAs) because they agglutinate RBCs at 4° C. Pascual, V., et al., (1991) J. Immunol. 146, 4385-4391; Pascual, V., (1992) J. Immunol. 149, 2337-2344; Silberstein, L. E., et al., (1991) Blood 78, 2372-2386. The ligands recognized by CAs are linear or branched glycoconjugates

The phrase “functionally neutralize circulating endotoxin” refers to having the property of preventing or inhibiting the proinflammatory actions of endotoxin on monocytes, macrophages and other cell types in the body. Generally, binding of an endotoxin neutralizing agent to endotoxin can result in prevention or inhibition of the proinflammatory actions of endotoxin. However, as used herein, binding of endotoxin is distinguished from functionally neutralizing endotoxin in that endotoxin neutralizing agents act by preventing the initiation of inflammatory responses, while endotoxin binding agents may or may not have this property. Preferably, functionally neutralizing endotoxin results in the clearance of endotoxin from circulation via a biological mechanism of immune complexation and adherence, for example, through the reticulendothelial system (RES) or via direct internalization by peripheral blood polymorphonuclear leukocytes. See for example Krieger, J. I., et al. (1993) J. Infect. Disease 167, 865-875. However, direct removal of endotoxin from a patient's blood (e.g., plasmapheresis) can have the effect of functionally neutralizing endotoxin.

The terms “level,” “amount” and “concentration” are used as commonly understood in the art of medicine and biochemistry. These terms are used interchangeably throughout the present disclosure.

The phrase “controlling endotoxemia” refers to maintaining the level of endotoxin in a patient's blood below a plasma concentration of about 100 pg/ml, more preferably below the threshold endotoxin concentration for endotoxemia (e.g., about 5-20 pg/ml), and most preferably at undetectable levels.

The term “specific binding” refers the property of having a high binding affinity of at least 10⁶ M⁻¹, and usually between about 10₆ M⁻¹ and about 10₉ M⁻¹. For example, the endotoxin neutralizing agent HA-1A (also referred to as “A6(H4C5)”), binds lipid A and the J5 mutant of E. coli 0111:B4 (lacking the O-specific side chain) with a binding affinity determined to be 3×10⁸M⁻¹. Bhat, N. M., et al. (1993) J. Immunol. 151, 5011-5021.

The term “therapeutically effective amount” is used to refer to an amount of an active agent having the ability to functionally neutralize circulating endotoxin in a patient resulting in clinical benefit as determined by reduced risk of shock, MODS, SIRS and mortality. In particular aspects, the therapeutically effective amount refers to a target serum concentration that has been shown to be effective in, for example, slowing or preventing disease progression, or hastening recovery. Efficacy can be measured in conventional ways, depending on the condition to be treated. For example, in patients with peritonitis due to ruptured viscus, efficacy can be measured by assessing the proportion of patients developing septic shock, or the proportion of patients dying, as well as by assessing renal, liver and cardiovascular function.

The terms “treat,” “treatment” and “therapy” and the like are meant to include therapeutic as well as prophylactic, or suppressive measures for a disease or disorder leading to any clinically desirable or beneficial effect, including but not limited to alleviation of one or more signs or symptoms, regression, slowing or cessation of progression of the disease or disorder. Thus, for example, the term treatment includes the administration of an agent prior to or following the onset of a sign or symptom of a disease or disorder thereby preventing or removing all signs or symptoms of the disease or disorder. As another example, the term includes the administration of an agent after clinical manifestation of the disease to combat the signs and symptoms of the disease. Further, administration of an agent after onset and after clinical signs or symptoms have developed where administration affects clinical parameters of the disease or disorder, such as the degree of tissue injury or mortality, whether or not the treatment leads to amelioration of the disease, comprises “treatment” or “therapy’ within the context of the invention.

The phrase “elevated calcitonin precursors” refers to concentrations of calcitonin precursors that are above the 95% confidence interval for the normal range obtained for healthy individuals. Normal levels of calcitonin precursors are in the range of 1.5-12 fmol/ml, as reported by Ammori, B. J., et al. (2003) Pancreas 27, 239-243 Calcitonin precursors can be determined using, for example, the radioimmunoassay described by Ammori, B. J., et al. or by ELISA.

The term “endotoxin” generically refers to the heat stable lipopolysaccharides (LPS) present in the outer membrane of certain gram negative bacteria. Endotoxin is reputed to exert its toxic effects via binding to CD14 and TLR-4 on macrophages and other cell types, thereby triggering the cascade of secretion of proinflammatory cytokines.

The term “endotoxemia” refers to conditions associated with serum endotoxin levels above a given threshold value. Endotoxemia is associated with detectable endotoxin levels of at least 5 pg/ml, generally at least 5-100 pg/ml, and more typically, endotoxin concentrations above 15-20 pg/ml.

The term “adult respiratory distress syndrome” (“ARDS”) refers to acute lung injury characterized by diffuse pulmonary inflammation, increased lung capillary permeability, pulmonary edema and respiratory insufficiency.

The term “multiple organ dysfunction syndrome” (“MODS”) refers to the refractory inability to maintain organ homeostasis in the absence of medical intervention. MODS can include acute renal failure (ARF), acute respiratory distress syndrome (ARDS), hepatobiliary dysfunction (HBD), central nervous system dysfunction (CNSD) or disseminated intravascular coagulation (DIC), among others.

The term “systemic inflammatory response syndrome” (“SIRS”) refers to the general systemic inflammatory response characterized by hyper- or hypothermia, tachycardia, hypoventilation and/or leukocytosis.

The term “sepsis” refers to a systemic response to a culture-documented infection consisting of two of the following four criteria: temperature >38° C. or <36° C.; heart rate >90 beats per minute; respiratory rate >20 breaths/minute; white blood cell count >12,000 cells/mm².

The term “septic shock” is a subset of sepsis and is defined as “sepsis-induced hypotension, persisting despite adequate fluid resuscitation, along with the presence of hypoperfusion abnormalities or organ dysfunction.”

The term “meningococcemia” refers to the acute systemic inflammatory syndrome marked by meningococcal bacteremia, septic shock and high mortality caused by the organism Neisseria meningitides.

The term “leaky gut syndrome”, “leaky bowel syndrome”, “bowel edema” or: leaky gut” are used interchangeably and refer to conditions wherein abnormal amounts of endotoxin are permitted to leak across the gastrointestinal mucosa into systemic circulation. This condition may exist as the result of many different types of systemic illness or injury such as infection, trauma, post-surgical state, or MODS.

Applicant has discovered that previous attempts to provide treatment of septic patients or patients suspected of bacteremia were flawed in providing an endotoxin neutralizing agent to patients without documented endotoxemia. Large controlled clinical trials of such patients concluded that there was no significant benefit in administering an endotoxin binding antibody to patients suspected of having gram negative bacteremia. Further, Applicant has discerned from these clinical trials that the early intervention with an endotoxin neutralizing agent in patients documented to have endotoxemia prior to treatment is critical to preventing progression of endotoxemia to sepsis and septic shock, SIRS, MODS, and mortality, while eliminating the risk of excess morbidity and mortality of administration of endotoxin neutralizing agents to patients without endotoxemia.

Accordingly, in one embodiment, methods for selecting a patient suffering from endotoxemia for treatment with an endotoxin neutralizing agent are provided comprising: (a) determining the level of endotoxin in the patient's blood; and (b) comparing the endotoxin level in the patient's blood to a predetermined threshold endotoxin level to determine if the patient has elevated endotoxin levels. The methods can further comprise treating patients identified as having elevated levels of endotoxin with an endotoxin neutralizing therapy selected from administering a pharmaceutical composition comprising a therapeutically effective amount of an endotoxin neutralizing agent to the patient, or performing plasmapheresis on the patient's blood using a plasmapheresis system comprising an endotoxin binding agent for removing endotoxin from the patient's blood, or combinations thereof. Preferably, the patient is a mammal and more preferably, the patient is a human.

In another aspect, methods are provided for treating a patient suffering from endotoxemia comprising: (a) determining the level of endotoxin in the patient's blood; (b) comparing the endotoxin level in the patient's blood to a predetermined threshold endotoxin level to determine if the patient has elevated endotoxin levels; and (c) treating patients identified as having elevated levels of endotoxin with an endotoxin neutralizing therapy selected from administering a pharmaceutical composition comprising a therapeutically effective amount of an endotoxin neutralizing agent to the patient, or performing plasmapheresis on the patient's blood using a plasmapheresis system comprising an endotoxin binding agent for removing endotoxin from the patient's blood, or combinations thereof.

In an additional aspect, methods are provided for controlling endotoxemia in a patient comprising: (a) determining the level of endotoxin in the patient's blood; (b) comparing the endotoxin level in the patient's blood to a predetermined threshold endotoxin level to determine if the patient has elevated endotoxin levels; and (c) treating patients identified as having elevated levels of endotoxin with a means for controlling endotoxemia. Preferably, the means for controlling endotoxemia comprises the administration of an endotoxin neutralizing agent or plasmapheresis to reduce the level of endotoxin in the patient's blood, or combinations thereof. Preferably, the level of endotoxin in the patient's blood is reduced below 100 pg/ml, more preferably to below about 5-20 pg/ml and most preferably to below the threshold of detection. The endotoxin neutralizing agent can be administered as a bolus or continuously over time. Preferably, the endotoxin level in the patient is maintained at or below the threshold level for endotoxemia. More preferably, the endotoxin level is reduced to undetectable levels.

In another aspect, methods are provided for preventing septic shock, SIRS, MODS or mortality in a patient comprising: (a) determining the level of endotoxin in the patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin.

In an additional aspect, methods are provided for reducing patient mortality, comprising: (a) determining the level of endotoxin in a patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin.

In yet another aspect, methods are provided for reducing hospital and/or intensive care unit duration comprising: (a) determining the level of endotoxin in a patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin. Preferably, the reduced hospital or intensive care unit duration is at least 0.5 days, more preferably one or more days, and most preferably at least two days reduced duration.

In other aspects, methods are provided for reducing the incidence of morbidities in a patient, comprising: (a) determining the level of endotoxin in the patient's blood; (b) comparing the endotoxin level in the patient's blood to a predetermined threshold endotoxin level to determine if the patient has elevated endotoxin levels; and (c) administering an endotoxin neutralizing agent to patients identified as having elevated levels of endotoxin. Typical morbidities include ARDS, SIRS, hepatic failure, renal failure, cardiac failure and MODS.

In another aspect, methods are provided for treating a patient suffering from endotoxemia, comprising: (a) determining the level of endotoxin in the patient's blood; (b) comparing the endotoxin level in the patient's blood to a predetermined threshold endotoxin level in the patient's blood to determine if the patient has elevated endotoxin levels; and (c) performing plasmapheresis on the patient's blood using a plasmapheresis system comprising an endotoxin binding agent for removing endotoxin from the patient's blood.

In yet other aspects, methods are provided for preventing septic shock, SIRS, MODS or mortality in a patient comprising: (a) determining the level of calcitonin precursors in the patient's blood; (b) comparing the level of calcitonin precursors in the patient's blood to a predetermined threshold level of calcitonin precursors in the patient's blood to determine if the patient has elevated calcitonin precursor levels; and if elevated, then measuring endotoxin levels in the blood of patients having elevated calcitonin precursors; and (c) administering an endotoxin neutralizing agent to patients identified as having elevated levels of calcitonin precursors and endotoxin.

In other aspects, methods are provided for reducing the risk of septic shock, SIRS, MODS or mortality in a patient suffering from pancreatitis or a condition characterized by leaky bowel syndrome or failure of gut barrier function, comprising: (a) determining the level of calcitonin precursors in the patient's blood; (b) comparing the level of calcitonin precursors in the patient's blood to a predetermined threshold calcitonin precursors in the patient's blood to determine if the patient has elevated calcitonin precursor levels; and if elevated, then measuring endotoxin levels in the blood of patients having elevated calcitonin precursors; and (c) administering an endotoxin neutralizing agent to patients identified as having elevated levels of calcitonin precursors and endotoxin.

In additional aspects, methods are provided for treating a patient at risk for developing septic shock, SIRS, MODS or mortality, comprising: (a) determining the level of endotoxin in the patient's blood; (b) comparing the endotoxin level in the patient's blood to a predetermined threshold endotoxin level in the patient's blood to determine if the patient has elevated endotoxin levels; and (c) administering a polyclonal antibody composition having binding specificity for at least one endotoxin epitope to patients identified as having elevated levels of endotoxin. Preferably, the polyclonal antibody composition has binding specificity for more than one endotoxin epitope.

II. Providing Safer Endotoxin Neutralizing Therapies

Excess mortality (relative to placebo) was seen in patients without documented gram negative bacteremia treated with anti-endotoxin antibodies in clinical studies. In one study, an excess mortality of 4% was observed, suggesting that 4% of patients would have survived if they had not been treated with the anti-endotoxin antibodies. Thus, by selecting only endotoxemic patients to receive an endotoxin neutralizing agent, the methods described herein provide increased safety relative to previously practiced methods because patients with no opportunity for improvement are not subjected to treatment with endotoxin neutralizing agents. Further, by selecting patients for treatment based on the patient's blood levels of endotoxin, i.e., not waiting for symptoms of sepsis, shock, or multi-organ failure to appear before beginning treatment, treatment can begin early enough to provide amelioration or prevention of the pro-inflammatory effects of endotoxemia.

Accordingly, methods for treating a patient suffering from a condition characterized by elevated levels of plasma endotoxin are provided, comprising: (a) determining the level of endotoxin in the patient's blood; (b) comparing the endotoxin level in the patient's blood to a predetermined threshold endotoxin level in the patient's blood to determine if the patient has elevated endotoxin levels; and (c) administering an endotoxin neutralizing agent to patients identified as having elevated levels of endotoxin, said method providing a reduced risk of sepsis, septic shock, SIRS, MODS or mortality and having improved safety relative to a method for administering an endotoxin neutralizing agent in the absence of elevated levels of plasma endotoxin. The methods provide an enhanced safety margin for administering the endotoxin neutralizing agent, resulting in reduced the risk of adverse events and mortality to the patient, because the endotoxin neutralizing agent is administered to the patient based on an objective measurement of a need for additional endotoxin neutralizing agent, and not on severity of illness or documented infection or other signs unrelated to endotoxemia.

Further, administering the endotoxin neutralizing agent based upon continued monitoring of the level of endotoxemia provides enhanced safety of administration of the agent in that the amount of agent administered is adjusted in a manner specific to the individual patient's need for endotoxin neutralization. This approach to treatment of endotoxemia can result in less overall exposure of the patient to the endotoxin neutralizing agent, thereby providing enhanced safety of administration of the agent.

III. Determination of Endotoxemia

Endotoxin concentrations can vary widely in individuals depending on health status, presence of stresses such as intestinal hypoxia or hemorrhagic shock, acute conditions such as pancreatitis, chronic conditions such as periodontitis, presence of infections, liver competency, etc. Otherwise healthy individuals have been observed with transient mild endotoxemia (increased endotoxin concentrations of 5-15 pg/ml) after physical stress such as athletic competition of long duration. Camus, G., et al. (1997) Clin. Sci (Lond.) 92, 415-422; Jeukendrup, A. E., et al. (2000) Clin. Sci (Lond.) 98, 47-55. Patients exhibiting varying degrees of periodontitis were shown to have increased serum endotoxin levels after gentle mastication (3.0+/−5.8 pg/ml) relative to levels observed before mastication (0.89+/−3.3 pg/ml). Geerts, S. O., et al. (2002) J. Periodontol. 73, 73-78.

However, endotoxin levels have been observed to vary widely in some critically ill patients. ICU patients fulfilling criteria for severe sepsis or septic shock have been observed with higher endotoxin levels of 310+/−810 pg/ml and 470+/−57 pg/ml. However, critically ill patients not diagnosed with sepsis also showed elevated endotoxin levels of 157 +/−140 pg/ml, but no patients with gram negative infection exhibited an endotoxin level below 50 pg/ml. Venet, C., et al. (2000) Intensive Care Med. 26, 538-544; Marshall, J. C., et al. (2002) Crit. Care 6, 289-290. Endotoxin levels in patients suffering from systemic meningococcal disease reached levels greater than 700 pg/ml, and were associated with increased development of septic shock, adult respiratory distress and death. Thus, endotoxin concentrations are elevated to varying degrees in patients suffering from a variety of diseases and conditions.

Endotoxemia as used herein is generally associated with detectable endotoxin levels of at least 5 pg/ml, generally in the range of 5 to at least 100 pg/ml. Endotoxin can be assayed, for example, by the chromogenic limulus amoebocyte lysate assay (Prior R. B. et al. (1979) J. Clin. Microbiol. 10, 394-395, and Hurley, J. C., et al., (1991) J. Clin Pathol. 44, 849-854), by the chemiluminescent assay as described in U.S. Pat. Nos. 5,804,370 and 6,159,683 to Romaschin, et al., as well as by ELISA or radioimmunoassay, without limitation, so long as the method provides sufficient sensitivity to measure the endotoxin. Endotoxin is preferably assayed using the endotoxin activity assay (EAA™) (Spectral Diagnostics, 135 The West Mall, Toronto, Canada). Marshall, J. C., et al. (2002) Crit. Care 6, 289-290. However, any sensitive and specific assay for endotoxin can be utilized.

Preferably, the endotoxin assay can be performed within 60 minutes or less, and is capable of providing information regarding endotoxin concentrations for a patient in real time, i.e., with minimal lag time (<60 minutes) between sample acquisition and determination of the endotoxin concentration in the sample.

In particular embodiments of the methods disclosed herein, the endotoxemia is associated with gram negative bacteremia. In additional embodiments, the endotoxemia is associated with gram positive bacteremia or fungemia. In certain other embodiments, the endotoxemia is present without documentable bacteremia or fungemia. In yet other embodiments, the endotoxemia is associated with infection with meningococcus. In additional embodiments, the endotoxemia is associated with a biowarfare agent, such as Yersinia pestis, Franciella tularensis, Shigella sp., Salmonella sp., or other gram negative bacteria. In alternative embodiments, the endotoxemia can be associated with neutropenia or other immune suppression. In yet other embodiments, the endotoxemia is associated with liver disease or pancreatitis. In still other embodiments, the endotoxemia is associated with bowel edema/leaky gut due to severe systemic illness such as infection, trauma, ischemia, chemotherapy, radiation therapy, post-surgical state, or multiorgan dysfunction syndrome (MODS), for example, where the multiorgan dysfunction syndrome is due to liver, renal, cardiac or lung failure. In additional embodiments, the endotoxemia is associated with peritonitis, neutropenia, urosepsis, severe liver injury, severe pancreatitis, leaky bowel syndrome, or meningococcemia.

IV. Active Agents

In a first aspect, active agents are provided that act as endotoxin neutralizing agents. Any endotoxin binding agent that can functionally neutralize the inflammatory activities of endotoxin can be utilized. The endotoxin neutralizing agent can be an antibody such as a monoclonal or polyclonal antibody that binds endotoxin. Preferably, the monoclonal antibody that binds endotoxin is HA-1A (U.S. Pat. No. 5,426,046, ATCC® Number HB-8669™), mAb 216, or E5 (U.S. Pat. No. 4,918,163, ATCC® Number HB-9081™), or a fragment, a fusion protein, a chimera, recombinant versions thereof, or combinations thereof. In a preferred embodiment, the monoclonal antibody that binds endotoxin can be a VH4-34 antibody. In further embodiments, the monoclonal antibody is an IgM or an IgG, or a fragment, a fusion protein, a chimera, recombinant versions thereof, or combinations thereof. The polyclonal antibody can be, for example, Pentaglobin®, or purified antibodies obtained from serum or recombinant methods In additional embodiments, the endotoxin neutralizing agent is a LPS binding protein, a bactericidal permeability increasing protein, or the like.

In additional aspects, endotoxin neutralizing agents can comprise pharmaceutically active agents that act to inhibit the proinflammatory actions resulting from the binding of endotoxin to a cellular receptor for endotoxin, for example, by blocking the binding of endotoxin to a cellular receptor for endotoxin or blocking signaling upon binding of endotoxin to a cellular receptor. In certain embodiments, the endotoxin neutralizing agent includes antagonists of TLR-4 receptor mediated inflammation, CD14, and the like. Preferably, the endotoxin neutralizing agent does not interfere with the binding of endogenous or exogenous anti-endotoxin antibodies to endotoxin that may be present in the blood of the patient.

Preferred endotoxin neutralizing agents include, without limitation, monoclonal or polyclonal antibodies, (including fragments, chimeras or fusion proteins thereof, and wherein the antibodies can be humanized, chimerized or produced by hydridomas or are recombinantly produced) bactericidal permeability inducing protein (BPI), lipopolysaccharide binding protein (LBP), and the like. Preferably, the endotoxin neutralizing agents are able to bind specifically to endotoxin and prevent the proinflammatory effects triggered by the presence of this molecule in the patient's body.

In a preferred embodiment, the endotoxin neutralizing agent is an antibody that binds endotoxin, and can be an antibody such as polyclonal antibody or monoclonal antibody. Antibodies that bind endotoxin are known in the art, and include HA-1A, E5 (Xomen), Pentaglobin® (Biotest, Frankfurt, Germany) and the like. HA-1A is a human IgM monoclonal antibody that was developed from a heterohybridoma created from spleen cells of a patient who had been immunized against the J5 mutant of Escherichia coli 0111:B4 (lacking the O-specific side chain). Teng, N. N. H., et al. (1985) Proc. Natl. Acad. Sci. USA 82, 1790-4. HA-1A binds lipid A and the J5 mutant of E. coli with a binding affinity determined to be 3×10⁸ M⁻¹. Bhat, N. M., et al. (1993) J. Immunol. 151, 5011-5021. E5 is a murine IgM monoclonal antibody raised in mice immunized against Escherichia coli J5 that binds to epitope on lipid A. Greenman, R. L., et al. (1991) J. Amer. Med. Assoc. 266, 1097-1102. Pentaglobin is a preparation of immunoglobulins purified from human plasma, possessing binding capacity for a variety of bacterial pathogens.

In additional preferred embodiments, the antibody that binds endotoxin possesses the property of activating complement and binding to Fc receptors, thereby facilitating clearance via the RES. In particular embodiments, the antibody is a recombinant IgG₁ or IgG₃, which are the IgG subclasses having the greatest complement fixing and Fc receptor binding activities, characteristics making them preferred embodiments for removing endotoxin from the blood by immune clearance mechanisms. In certain embodiments, the recombinant IgG₁ or IgG₃ antibody comprises at least one CDR sequence derived from HA-1A or E5.

Bactericidal/permeability-increasing protein (“BPI”) is a human host-defense protein made by polymorphonuclear leukocytes (PMNL). BPI has been reported to kill gram-negative bacteria, enhance the activity of antibiotics, neutralize gram-negative endotoxin and inhibit angiogenesis. BPI includes recombinant versions such as rBPI21 developed at Xoma.

LBP is a glycoprotein present at concentrations of less than 100 ng/ml in the serum of healthy animals and man, but can reach concentrations of 30-50 μg/ml. Tobias, et al. (1986) J. Exp. Med. 164, 777-793. LBP recognizes the lipid A region of LPS and forms high affinity, 1:1 stoichiometric complexes with both rough and smooth form LPS. Tobias, et al. (1989) J. Biol. Chem. 264, 10867-10871. LBP bears N-terminal sequence homology with BPI. Tobias, et al. (1988) J. Biol. Chem. 263, 13479-13481. LBP is not directly cytotoxic for gram-negative bacteria (Tobias, et al. (1988) J. Biol. Chem. 263,13479-13481).

The endotoxin neutralizing agent can be an antibody such as a monoclonal or polyclonal antibody that binds endotoxin. Preferably, the monoclonal antibody that binds endotoxin is HA-1A, mAb 216, or E5, or a fragment, a fusion protein, a chimera, humanized versions thereof, recombinant versions thereof, or combinations thereof. In a preferred embodiment, the monoclonal antibody that binds endotoxin can be a VH4-34 antibody. In further embodiments, the monoclonal antibody is an IgM or an IgG, or a fragment, a fusion protein, a chimera, recombinant antibody or combinations thereof. In particular embodiments, the antibody is an IgG₁ or an IgG3. The polyclonal antibody can be, for example, Pentaglobin or purified antibodies obtained from serum or recombinant methods In additional embodiments, the endotoxin neutralizing agent is a LPS binding protein, a bactericidal permeability increasing protein, or the like.

Additional active agents include those utilized to treat febrile patients, bacteremic patients, fungemic patients, or patients suffering from leaky bowel syndromes, or other patients at risk for deleterious clinical consequences of having endotoxin present in their blood. Typical additional active agents include antibiotics, anti-inflammatory agents such as nonsteroidal anti-inflammatory drugs (“NSAIDs”); anti-inflammatory steroids; Toll-like receptor 4 (“TLR-4 receptor antagonists”), anticoagulants, and cytokine inhibitors, such as IL-6 inhibitors, IL-1 inhibitors, TNF inhibitors, and the like. The additional active agent can also include agents such as A1 adenosine receptor antagonists such as 1,3-dipropyl-8-cyclopentylxanthine (DPCPX) and bamiphylline and others described in U.S. Patent Application Publication No. 20060276378, which are purportedly useful for blocking endotoxin-induced acute lung injury in animals.

Antibiotics useful as additional active agents are well known in the art. Some representative antibiotics include sulfonamides, such as sulfisoxazole or sulfadoxine and the like, optionally with trimethoprim; quinolones, such as nalidixic acid, and fluoroquinolones such as ciprofloxacin and ofloxacin and the like; penicillins such as penicillin G or V, and semi-synthetic penicillins such as the isoxazolyl penicillins oxacillin, cloxacillin and dicloxacillin and the like, aminopenicillins such as ampicillin, amoxicillin and their congeners, antipseudomonal penicillins including the carboxypenicillins carbenicillin and ticarcillin and the ureidopenicillin mexlocillin and the like; cephalosporins and cephamycins such as cephalosporin C, cephalothin, cefamandole, cefotaxime, cefepime, and cefotetan and the like; carbapenems such as imipenem, meropenem, aztreonam and the like, optionally in combination with β-lactamase inhibitors such as clavulinic acid; aminoglycosides such as streptomycin, tobramycin, kanamycin and gentamycin and the like; tetracyclines such as chlortetracycline, doxycycline, and the like; chloramphenicol; macrolides such as erythromycin, clarithromycin and azithromycin and the like as well as clindamycin; streptogramins A and B, such as quinupristin and dalfopristin; oxazolidinones such as linezolid; aminocyclitols such as spectinomycin; polymyxins such as polymyxin BI and colistin; glycopeptides such as vancomycin, daptomycin, LY 333328, teicoplanin and the like; polypeptide antibiotics such as bacitracins; antimycobacterial agents such as nicotinamides (e.g., isoniazid and pyrazinamide) and macrocyclic antibiotics (e.g., rifampins); antifungal agents such as the polyene macrolides (e.g., amphotericins A and B), fluorinated pyrimidines such as flucytosine, azole antifungals including imidazoles such as clotrimazole, miconazole, ketoconazole, econazole, butoconazole, oxiconazole and sulconazole, and triazoles terconazole, fluconazole and itraconazole, pneumocandins, griseofulvin, terbinafine, and the like. Preferred antibiotics are selected from the cephalosporins, aminoglycosides, semi-synthetic penicillins and quinolones.

In addition, antibiotics can include those from new emerging classes of antibiotics including, inhibitors of DNA methyltransferase, pyrrole tetramide DNA binders, heteroaromatic polycyclic (HARP) compounds, anti-bacterial DNA binders, benzamides, benzothiophenes, isoquinoline analogs, gyrase inhibitors, pyrido[1,2-c]pyrimidine gyrase inhibitors, benzimidazole/benzoxazole gyrase inhibitors, quinazolinedione gyrase inhibitors, PcrA inhibitors, inhibitors of RNA polymerase, RNA bacterial ribosome targets, including protein synthesis inhibitors, inhibitors of RNA-protein interactions (complexes), transcriptional/translational inhibitors, paromomycin, elongation inhibitors, translation inhibitor TAN-1057, aminoacyl-tRNA synthetase inhibitors, chuangximycin analogs, peptide deformylase (PDF) inhibitors, bacterial cell wall inhibitors, including UDP-N-aceytylmuramate/L-alanine ligase (Mur C) and other mur (D and I) inhibitors, as well as phosphor-N-acetylmuramyl-pentapeptide translocase (Mra Y), including muraymycin C₁, muraymycin A1, mureidomycin A, liposidomycin C, RU75411, penicillin binding protein (PBP) inhibitors, inhibitors of bacterial cell membranes, including inhibitors of lipid A biosynthesis, metalloenzyme inhibitors, hydroxamic acid inhibitors, including BB-78484, BB-78485, inhibitors of 3-deoxy-D-manno-2-octulosonate-8-phosphate synthetase (KDOP), mutulin derivatives, althiomycin and analogs of althiomycin, naphthyridine agents, pyrimidine-pyridine analogs, piperidine agents, tetrahydroquinoline analogs, mannopeptimycin, AC-98-5, AC-98-6446, phosphoryl transfer system (PTS) inhibitors, AI-2 signaling pathway inhibitors, dehydroquinate synthetase (DHQS) inhibitors, Ar-358, shikimate kinase inhibitors, chorismate synthase (CS) inhibitors, PTX110130, PTX008313, nicotinamide adenine dinucleotide (NAD) synthetase inhibitors, fatty acid biosythesis inhibitors, thiolactomycin, triclosan, cerulenin, phosphopantetheine adenylyltransferase (PPAT) inhibitors, PTX-042695, PTX-031553, PTX-007063, PTX-008134, Fab inhibitors, f-ketoacyl-acyl carrier protein (ACP) synthase III (FABH) inhibitors, thiolactomycin analogs, enoyl-ACP reductase (FabI or FabK) inhibitors, SB-627696, SB-663042, and SB-633857, and the like.

Some nonlimiting examples of NSAIDs include salicylic acid derivatives such as aspirin, sulfasalazine, salicylamide, sodium salicylate, and salicylate potassium; aryl propionic acids including benoxaprofen, decibuprofen, flurbiprofen, fenoprofen, ibuprofen, indoprofen, ketoprofen, naproxen, naproxol, oxaprozin; heteroaryl acetic acids such as diclofenac, ketorolac, tolmetin; indole and indene acetic acids including indomethacin, sulindac; selective COX-2 inhibitors such as celecoxib, rofecoxib, valdecoxib, etodolac, ibufenac, nimesulfide; alkanones such as nabumetone; oxicams including meloxicam, piroxicam, lomoxicam, cinnoxicam, sudoxicam, tenoxicam; anthranilic acids such as mefenamic acid and meclofenamic acid The dose of NSAID is typically 0.5 mg to 1000 mg, but can be higher as tolerated by the patient. NSAIDs can be administered by any convenient method, including intravenous administration.

TLR-4 receptor antagonists include, without limitation, E5564, B531, or B1287, etc. (Eisai Company Ltd), TAK-242 (Takeda Pharmaceuticals), compounds purportedly useful in a method of mitigating or preventing an acute phase inflammatory response associated with endotoxic shock syndrome as described in U.S. Patent Application Publication No. 20060211752 to Kohn, et al., for example a therapeutically effective amount of one or more compound selected from methimazole (MMI), phenylmethimazole, and tautomeric cyclic thione compounds and active derivatives thereof capable of preventing, ameliorating or inhibiting pathologies that are mediated or associated with Toll-like receptor 3 or Toll-like receptor 4 overexpression, activation, and signaling or both together.

Anticoagulants typically, include heparin, and an activated Protein C such as drotrecogin-alpha (activated), which is a genetically engineered Protein C sold under the brand name Xygris (Eli Lilly & Co.). Nonlimiting examples of anti-inflammatory steroids include for example prednisolone, prednisone, methylprednisolone, triamcinolone or dexamethasone). Additional active agents can include treatment with inhibitors of proinflammatory cytokines, i.e., cytokine inhibitors, such as inhibitors of TNF, IL-1, or IL-6, etc. 5-lipoxygenase inhibitors, LTB₄ antagonists and LTA₄ hydrolase inhibitors and anti-cell adhesion molecules, such as anti E-selectin.

The term “conjugate” refers to coupling of active agents, which can be covalent or noncovalently associated, and includes immunoconjugates or conjugates of other active agents. Conjugates can be prepared from endotoxin neutralizing agents in particular, including antibodies (and derivatives thereof such as recombinant versions thereof, fragments, chimeras, fusion proteins, etc.), LPS binding proteins, bactericidal permeability inducing protein, polymyxin, and the like. Immunoconjugates are conjugates of antibodies to active agents, and include therapeutic compositions or diagnostic compositions useful in monitoring the efficacy of treatment, for example, such as conjugates comprising indicator molecules such as colloidal beads, fluorescent dyes, enzymes, radioisotopes, and the like.

Conjugates can be prepared by numerous methods known in the art, such as chemical derivatization of the active agent to provide reactive crosslinking groups, which can be labile or non-labile. Labile reactive groups provide for the release of the label from the active agent. Non-labile crosslinking is also useful. Conjugation can be achieved by a variety of means known to the art including conventional coupling techniques (e.g., coupling with dehydrating agents such as dicyclohexylcarbodiimide (DCCI), ECDI and the like), the use of linkers capable of coupling through sulfhydryl groups, amino groups or carboxyl groups (available from Pierce Chemical Co., Rockford, Ill.), by reductive amination.

In one method, an immunoconjugate, can be prepared by first modifying the antibody with a cross-linking reagent such as N-succinimidyl pyridyldithiopropionate (SPDP) to introduce dithiopyridyl groups into the antibody (Carlsson et al. (1978) Biochem. J. 173, 723-737; U.S. Pat. No. 5,208,020). In a second step, an agent having a thiol group, is added to the modified antibody, resulting in the displacement of the thiopyridyl groups in the modified antibodies, and the production of disulfide-linked agent-antibody conjugate. A similar approach can be used to generate conjugates of LBP or BPI, for example.

V. Therapeutic Plasmapheresis

Plasmapheresis and immunoadsorption procedures and devices are known in the art, and typically involve the separation of plasma from cellular blood components using centrifugation. Instruments can be calibrated to perform plasmapheresis, plateletpheresis (collection of donor platelets for patient use), erythrocytopheresis (used for treatment of sickle cell anemia), or leukopheresis (collection of donor stem cells for transplantation; removal of white blood cells for therapeutic purposes). Differential cell density gradients allow centrifugal separators to apherese by continuous or discontinuous methods. Hollow fiber or rotating cylinder membranes can also be used to effect separation. Membranes can be used with a dialyzer or a centrifugation device to separate blood constituents using a filtration process, allowing lower molecular weight components to pass through the membrane while retaining higher molecular weight components. A typical membrane comprises cellulose acetate, although a variety of materials can be designed to selectively retain specific plasma components by cryoprecipitation (removal of cryoglobulins) or affinity adsorption (e.g., removal of IgG-class antibodies by adsorption to Staphylococcus protein A). Membranes can be utilized singly or multiply so that the first membrane separates plasma from cellular components and the second selectively removes specific plasma components.

The term immunoadsorbent is used in its broadest sense to refer to matrices capable of binding to a desired epitope with high binding affinity (i.e., specific binding) comprising filters, membranes, particles, beads, and the like, as well as monolithic materials. Immunoadsorbents derivatized with monoclonal antibodies provide a means for the highly specific removal of plasma constituents, including endotoxin and cytokines. Coupling techniques well known in the art can also be utilized to prepare immunoadsorbents having a desired specific binding. Immunoadsorbents can be utilized to remove endotoxin from the blood or plasma of a patient. Examples of preferred immunoadsorbents include endotoxin binding antibodies (e.g., HA-LA, mAb 216, E5) or fragments, chimeras or fusion poteins thereof, recombinant versions thereof, conjugated to membranes or other matrices.

In addition to immunoadsorbents, matrices capable of binding endotoxin (i.e., matrices derivatized with an endotoxin binding agent) can also be utilized in the present methods. For example, a polymyxin B adsorbent column utilizing immobilized fiber has been described and shown to remove endotoxin by direct hemoperfusion. Uriu, K., et al. (2002) Am. J. Kidney Dis. 39, 937-47. Additional examples of sorbents that can remove endotoxin include matrices derivatized with LPS binding protein, bacterial permeability inducing protein, and the like, without limitation. The particular endotoxin binding agent is not critical, so long as it binds endotoxin with sufficiently high affinity to effect its removal from the patient's blood.

Accordingly, methods are provided for treating a patient suffering from endotoxemia, comprising (a) determining the level of endotoxin in the patient's blood; (b) comparing the endotoxin level in the patient's blood to a predetermined threshold endotoxin level in the patient's blood to determine if the patient has elevated endotoxin levels; and (c) performing plasmapheresis on the patient's blood using a plasmapheresis system comprising an endotoxin binding agent for removing endotoxin from the patient's blood. In certain embodiments, the plasmapheresis system comprising an endotoxin neutralizing agent comprises a polymyxin, a bactericidal permeability inducing protein, a LPS binding protein, or one or more monoclonal or polyclonal antibodies exhibiting specific binding for endotoxin.

In a preferred embodiment, methods are provided for removing endotoxin from the body of a patient suffering from a condition characterized by elevated levels of endotoxin, comprising contacting the blood or plasma of the patient with an adsorbent (e.g., an immunoadsorbent) having specific binding for endotoxin, wherein said contacting results in the reduction in the amount of endotoxin present in the blood of the patient. Preferably, the adsorbent is an immunoadsorbent that comprises a monoclonal or polyclonal antibody, or fragments, chimeras, or fusion proteins thereof, or combinations thereof. In a preferred embodiment, the immunoadsorbent comprises HA-1A, Mab216 or E5, humanized or chimerized versions thereof, or fragments, or fusion proteins thereof, or combinations thereof. In certain embodiments, the immunoadsorbent comprises one or more VH4-34 antibodies. Preferably said contacting is effected using plasmapheresis.

VI. Methods for Monitoring the Efficacy of an Endotoxin Neutralizing Therapeutic Treatments

In one aspect, methods are provided for monitoring the therapeutic efficacy of a treatment for endotoxemia in a patient in need thereof, comprising: (a) determining the level of endotoxin in the patient's blood; (b) performing a treatment for endotoxemia; (c) determining the level of endotoxin in the blood of a patient at a time after the treatment for endotoxemia; (d) comparing the endotoxin level in the patient's blood to a predetermined threshold endotoxin level or the level of endotoxin prior to treatment to determine whether the endotoxin level in the patient's blood has decreased due to the treatment for endotoxemia or whether the endotoxin level remains elevated. Therapeutic treatments include plasmapheresis, treatment with an endotoxin neutralizing agent, treatment with an additional pharmaceutically active agent, or combinations thereof.

The methods can further comprise providing additional treatments for endotoxemia to patients identified as having elevated levels of endotoxin, for example, plasmapheresis or the administration of an endotoxin neutralizing agent, with or without additional active agents, or combinations thereof. Additional active agents include, but are not limited to, antibiotics, TLR-4 receptor antagonists, cytokine inhibitors, anti-inflammatory agents, or anticoagulants, or combinations thereof.

In an additional aspect, methods are provided for monitoring the therapeutic efficacy of a treatment for endotoxemia in a patient in need thereof, comprising: (a) determining the amounts of endotoxin and endotoxin neutralizing agent in the patient's blood; and (b) comparing the amount of endotoxin neutralizing agent to the amount of endotoxin in the patient's blood to determine if the patient has sufficient endotoxin neutralizing agent to functionally neutralize the endotoxin. The methods can further comprise (c) administering an endotoxin neutralizing agent to patients identified as having an insufficient amount of endotoxin neutralizing agent to functionally neutralize the endotoxin in the patient's blood, and the endotoxin neutralizing agent administered in step (c) can be the same or different from the endotoxin neutralizing agent present in the patient's blood. Preferred endotoxin neutralizing agents are selected from a LPS-binding protein, anti-endotoxin antibody, or bactericidal permeability inducing protein. In a particular embodiment, the anti-endotoxin antibody is an endogenous anti-endotoxin antibody or an exogenous anti-endotoxin antibody. In another embodiment, the anti-endotoxin antibody can be a VH4-34 antibody. In a preferred embodiment, the exogenous anti-endotoxin antibody is selected from HA-1A, E5, Mab 216, recombinant versions thereof, fragments, fusion proteins, chimeras, or combinations thereof.

In yet other aspects, methods are provided for monitoring the therapeutic efficacy of a treatment for endotoxemia in a patient in need thereof, comprising: (a) determining the amount of endotoxin neutralizing agent in the patient's blood; and (b) comparing the amount of endotoxin neutralizing agent in the patient's blood to the amount of endotoxin neutralizing agent that would be sufficient to functionally neutralize an endotoxin concentration of from about 5 to about 1000 pg/ml in the patient's blood to determine if the patient has sufficient endotoxin neutralizing agent in their blood to functionally neutralize an endotoxin concentration of from about 5 to about 1000 pg/ml. The methods can further comprise (c) administering an endotoxin neutralizing agent to patients identified as having an amount of endotoxin neutralizing agent in their blood that is not sufficient to functionally neutralize an endotoxin concentration of from about 5 to about 1000 pg/ml.

The monitoring of treatment efficacy can also include patient assessment measures that are well known in the art of medical diagnosis and practice. For example, the monitoring of treatment efficacy can include monitoring disease progression or amelioration of symptoms using various well known clinical signs such as temperature, heart rate, breathing rate; clinical activity scales; assays of organ function; levels of C-reactive protein (CRP) and cytokines, and the like.

VII. Methods for Determining an Effective Dose of an Endotoxin Neutralizing Agent

The amount of endotoxin in the patient's blood can be determined using an assay (e.g., an immunoassay or the Spectral chemiluminescent assay). The total amount of endotoxin can be calculated based on multiplying the blood endotoxin concentration times the estimated blood volume of the patient, or the estimated plasma volume of the patient using standard nomograms (based on the patient's weight to provide an estimate of the patient's total plasma volume).

The amount of endotoxin neutralizing agent to be administered or re-administered can be determined by dividing the total amount of endotoxin by the endotoxin neutralizing capacity per mg of the endotoxin neutralizing agent (e.g., antibody, LPS binding protein, etc.). The endotoxin neutralizing capacity can be estimated based on a stoichiometric calculation of moles endotoxin binding sites on the endotoxin neutralizing agent to endotoxin. For example, an anti-endotoxin IgM antibody can be assumed to have a stoichiometry of 1:1 with endotoxin even though as a pentamer theoretically it can bind approximately 10 endotoxin molecules, because once the antibody fixes complement, it is cleared by macrophages or the reticuloendothelial system. Hence, the calculation results in the total dose of endotoxin neutralizing agent required to neutralize total body endotoxin load. The treating physician then administers a total dose in excess of this amount and can periodically re-assay the patient's blood for the presence of endotoxin to determine the appropriate time interval and total dose of endotoxin neutralizing agent for re-administration.

The dosage of endotoxin neutralizing agent can vary from about 0.01 mg/m² to about 500 mg/m², preferably 0.1 mg/m² to about 200 mg/m², most preferably about 0.1 mg/m² to about 10 mg/m². Such dosages may vary, for example, depending on whether repeatedly or continuously administered to the patient, tissue, and other factors known to those of skill in the art. Preferably, the dosage is sufficient to neutralize endotoxin in the patient's blood. In a preferred embodiment, the dose of endotoxin neutralizing agent reduces the endotoxin concentration in the patient's blood to a level at or below the threshold level of about 5-20 pg/ml, and most preferably, below the level of detection.

VIII. Assays

Endotoxin, calcitonin precursores and endotoxin neutralizing agents can be detected when present in samples of biological fluids and tissues. Any sample containing a detectable amount of these compounds can be used. A sample can be a liquid such as urine, saliva, cerebrospinal fluid, blood, serum and the like, or a solid or semi-solid such as tissues, feces, and the like, or, alternatively, a solid tissue such as those commonly used in histological diagnosis. Preferably, the sample is blood or blood components such as plasma.

Standard biochemical assays can be utilized as a means for determining the concentration of endotoxin, calcitonin precursors, VH4-34 antibodies, endogenous or exogenous anti-endotoxin antibodies, etc. in a sample obtained from a patient. Various heterogenous and homogeneous protocols, either competitive or noncompetitive, can be employed. In particular, assays utilizing monoclonal antibodies can be employed in a competitive or non-competitive mode and in either a direct or indirect format. Examples of such assays are the radioimmunoassay (RIA) and the sandwich (immunometric) assay. A particularly preferred assay format is in an ELISA assay and utilizes capture and detection antibodies. This “sandwich” format is well known in the art, and can be practiced by one skilled in the art without undue experimentation. Detection can be effected utilizing immunoassays which are run in either the forward, reverse, or simultaneous modes, including immunohistochemical assays on physiological samples. Those of skill in the art will know, or can readily discern, other immunoassay formats without undue experimentation.

Endotoxin, calcitonin precursors and endotoxin neutralizing agents, as well as antibodies or other agents with binding specificity therefore, can be bound to many different carriers. Some nonlimiting examples of well-known carriers include glass, polystyrene, polypropylene, polyethylene, dextran, nylon, amylases, natural and modified celluloses, polyacrylamides, agaroses and magnetite. The carrier can be either soluble or insoluble depending on the assay format. Those skilled in the art will know of or will be able to ascertain other suitable carriers for binding endotoxin and endotoxin neutralizing agents using routine experimentation.

There are many different labels and methods of labeling known to those of ordinary skill in the art. The term “label” used in various grammatical forms refers to single atoms or molecules that are either directly or indirectly involved in the production of a detectable signal to indicate the presence of a complex. Any label can be linked to or incorporated in an expressed protein, polypeptide, or antibody molecule described herein, or used separately, and those atoms or molecules can be used alone or in conjunction with additional reagents. Examples of the types of labels which can be used in assays include enzymes, radioisotopes, fluorescent compounds, colloidal metals, chemiluminescent compounds, and bioluminescent compounds. Suitable fluorescent labeling agents are fluorochromes such as fluorescein isocyanate (FIC), fluorescein isothiocyante (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC), and the like. A description of immunofluorescence analysis techniques is found in DeLuca, “Immunofluorescence Analysis”, in Antibody as a Tool, Marchalonis et al., eds., John Wiley & Sons, Ltd., pp. 189-231 (1982), which is incorporated herein by reference.

Those of ordinary skill in the art will know of other suitable labels or will be able to ascertain such, using routine experimentation. Furthermore, the binding of these labels to endotoxin, calcitonin precursors and endotoxin neutralizing agents can be done using standard techniques common to those of ordinary skill in the art.

In preferred embodiments, the label is an indicating group such as an enzyme (e.g., horseradish peroxidase (HRP), glucose oxidase, or the like). In such cases where the indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-amino-di-(3-ethyl-benzthiazoline-G-sulfonic acid) (ABTS).

Radioactive elements are also useful labels. Suitable radiolabels include those producing gamma emissions, such as ¹²⁵I, ¹³¹I and ⁵¹Cr, or ¹¹C, ¹⁸F, ¹⁵O and ¹³N which emit positrons, and upon encounters with electrons present in the body decay with gamma emission. Also useful are beta emitters, such as ³H or ³²P.

Other labels include low molecular weight haptens that can be coupled to the compounds of interest, and can result in enhanced detection sensitivity. These haptens can then be specifically detected by means of a second reaction. For example, it is common to use haptens such as biotin, which reacts with avidin, or dinitrophenol, pyridoxal, or fluorescein, which can react with specific anti-hapten antibodies.

A. Assays for Endotoxin Neutralizing Agents

In one embodiment, the endotoxin neutralizing agent is an anti-endotoxin antibody (e.g., mAb 216, HA-1A). In a first aspect, the assay can involve binding LPS on the ELISA substrate (e.g., plate). The anti-endotoxin antibody, for example, HA-1A or E5, present in the sample will be bound to LPS on the ELISA plate and can be detected using an enzyme linked secondary antibody such as peroxidase labeled anti-IgM.

In another embodiment, the endotoxin neutralizing agent is a VH4-34 antibody (e.g., mAb 216, HA-1A). In a first aspect, the assay involves providing a capture antibody capable of binding the endotoxin neutralizing agent, in this embodiment, a VH4-34 antibody such as HA-1A. A suitable capture antibody capable of binding HA-1A is 9G4. By providing 9G4 on the ELISA substrate (e.g., plate), HA-1A or other VH4-34 antibody present in the sample can be bound to the ELISA plate and can be detected using an enzyme linked antibody such as peroxidase labeled anti-IgM.

ELISA can also be utilized to determine the amount of BPI or LBP present in a sample. For example, antibodies specific for LBP can be bound to an ELISA substrate and contacted with the plasma sample from a patient containing LBP as an endotoxin neutralizing agent, which will bind to the antibodies bound to the substrate. The presence of bound LBP can be detected by either of two methods: 1) by using a second antibody (labeled, for example, with an enzyme, fluorophore, radioisotope, etc.) having specific binding for a different epitope on LBP. The binding of this second antibody to LBP bound by the first antibody can be detected (e.g., by the enzyme reaction catalyzed by the label such as horseradish peroxidase oxidation of a chromogen or fluorophore, or by fluorescence of a fluorophore label or radioisotope, etc.) and indicate the presence of bound LBP. 2) A competition assay using labeled LBP can be used to detect the presence of the LBP bound by antibody. Antibodies to epitopes on LBP can be generated or are available. For example, murine antibodies specific for LBP have been described in U.S. Pat. No. 6,884,417 to Kirkland, incorporated by reference herein. These anti-LBP antibodies are secreted by hybridomas Mab 1E8 and 2B5, deposited with the American Type Culture Collection, 10801 University Blvd., Manassas, Va. 20110-2209 (ATCC), having respective ATCC Accession Numbers 11490 and 11491.

Assays for BPI are known in the art. For example, U.S. Pat. No. 6,759,203 to White, incorporated by reference herein, purports to disclose a sandwich ELISA assay for human BPI which exhibits high assay sensitivity, high specificity, and excellent reproducibility. BPI is preferably measured in plasma (the acellular fluid portion of blood obtained by adding anticoagulants (e.g., citrate, acid-citrate-dextrose (ACD), EDTA, heparin and hirudin) to prevent clotting, and not in serum, thereby avoiding artifacts caused by release from neutrophils. Alternatively, novel antibodies to BPI can be generated and utilized in ELISA assays as described above.

B. Assays for Endotoxin

Endotoxin can be assayed by any means known in the art, for example, by the chromogenic limulus amoebocyte lysate assay, by the chemiluminescent assay as described in U.S. Pat. Nos. 5,804,370 and 6,159,683 to Romaschin, et al., as well as by ELISA or radioimmunoassay, without limitation, so long as the method provides sufficient sensitivity to measure endotoxin present in plasma in concentrations of 5-100 pg/ml or higher. Patient samples can be diluted as necessary to provide endotoxin concentrations in the range suitable for the assay.

Endotoxin is preferably assayed using the endotoxin activity assay (EAA™) (Spectral Diagnostics, 135 The West Mall, Toronto, Canada). Marshall, J. C., et al. (2002) Crit. Care 6, 289-290. However, any sensitive and specific assay for endotoxin can be utilized. Preferably, the endotoxin assay can be performed within 60 minutes or less, and is capable of providing information regarding endotoxin concentrations for a patient in real time, i.e., with minimal lag time (<60 minutes) between sample acquisition and determination of the endotoxin concentration in the sample.

IX. Kits

In a further aspect, kits are provided for selecting a patient suffering from endotoxemia for treatment with an endotoxin neutralizing agent comprising: (a) means for determining the level of endotoxin in the patient's blood; and (b) instructions for determining whether the patient has elevated levels of endotoxin in their blood; and optionally, (c) at least one dose of a therapeutic composition comprising an endotoxin neutralizing agent.

In a further aspect, kits are provided for treating a patient suffering from endotoxemia comprising: (a) means for determining the level of endotoxin in the patient's blood; (b) instructions for determining whether the patient has elevated levels of endotoxin in their blood; and (c) at least one dose of a therapeutic composition comprising an endotoxin neutralizing agent.

In an additional aspect, a kit is provided for monitoring the therapeutic efficacy of a treatment for endotoxemia in a patient in need thereof, comprising: (a) means for determining the level of endotoxin in the patient's blood before and after performing a treatment for endotoxemia; (b) instructions for determining whether the patient has elevated levels of endotoxin in their blood; and optionally, (c) at least one dose of a therapeutic composition comprising an endotoxin neutralizing agent.

In a further embodiment, kits are provided for monitoring the therapeutic efficacy of a treatment for endotoxemia in a patient in need thereof, comprising: (a) means for determining the amounts of endotoxin and endotoxin neutralizing agent in the patient's blood; and (b) instructions for determining whether the patient has elevated levels of endotoxin in their blood and whether the patient has sufficient amount of endotoxin neutralizing agent to functionally neutralize the amount of endotoxin in their blood or an amount of endotoxin of between about 5 and at least about 1000 pg/ml.

In another aspect, kits are provided for preventing septic shock, SIRS, MODS or mortality in a patient comprising: (a) means for determining the level of calcitonin precursors and endotoxin levels in the patient's blood; (b) instructions for determining whether the patient has elevated calcitonin precursor and endotoxin levels; and optionally, (c) at least one dose of an endotoxin neutralizing agent.

In another aspect, kits are provided for reducing the risk of septic shock, SIRS, MODS or mortality in a patient suffering from pancreatitis or a condition characterized by leaky bowel syndrome or failure of gut barrier function, comprising: (a) means for determining the level of calcitonin precursors and endotoxin in the patient's blood; (b) instructions for determining if the patient has elevated calcitonin precursor and endotoxin levels; and optionally, (c) at least one dose of an endotoxin neutralizing agent.

Kits can include calibrated reagents for determining the level of calcitonin precursors, endotoxin and/or endotoxin neutralizing agent(s) in the patient's blood. Typically, the means for determining the level of calcitonin precursors includes ELISA assays, radioimmunoassay and the like. For example, a radioimmunoassay has been described by Ammori, B. J., et al. (2003) Pancreas 27, 239-243. Preferably, the means for determining the level of calcitonin precursors can be performed in a short period of time, preferably less than 6 hours, and more preferably less than 3 hours.

In a preferred embodiment, the kit includes a rapid endotoxin assay kit and a endotoxin neutralizing agent assay kit. The kit can further comprise at least one endotoxin neutralizing agent for administering to the patient.

Additional reagents can also be included in the kits as desired, for example, control antibodies, secondary antibodies, supplies for ELISA assays, radioimmunoassay, or the like.

X. Compositions

In another aspect, pharmaceutical compositions are provided for treating a patient suffering from endotoxemia, comprising an effective amount of at least one endotoxin neutralizing agent and a pharmaceutically acceptable carrier. In a particular embodiment, the composition comprises at least two endotoxin neutralizing agents. In another embodiment, the endotoxin neutralizing agent is at least one monoclonal antibody that binds endotoxin, such as, but not limited to, HA-1A, mAb 216, or E5, or fragments, fusion proteins, chimeras, or recombinant versions thereof, or combinations thereof. In a particular embodiment, the monoclonal antibody that binds endotoxin is a VH4-34 antibody. The monoclonal antibody can be an IgM or an IgG, preferably an IgG₁ or an IgG₃, or a fragment, a fusion protein, a chimera, or recombinant versions thereof, or combinations thereof.

In another embodiment, the at least one endotoxin neutralizing agent is a LPS binding protein or a bactericidal permeability increasing protein, and in certain embodiments, a TLR-4 receptor antagonist. In certain particular embodiments, the composition comprises at least two of the following: a monoclonal antibody that binds endotoxin, a LPS binding protein, a bactericidal permeability increasing protein, or combinations thereof. The composition can further comprise an additional active agent, such as, but not limited to, an antibiotic, a TLR-4 receptor antagonist, a cytokine inhibitor, an anti-inflammatory agent, or an anticoagulant, or combinations thereof. In a preferred embodiment, the composition comprises at least one endotoxin neutralizing agent and a TLR-4 receptor antagonist. In an additional embodiment, a composition is provided for treating a patient suffering from endotoxemia, comprising a polyclonal antibody composition having binding specificity for at least one endotoxin epitope. The polyclonal antibody composition can have binding specificity for more than one endotoxin epitope.

Endotoxin neutralizing agents, kits and antibodies can be prepared or formulated using any methods and pharmaceutically acceptable excipients known in the art. Typically, antibodies are provided in saline, with optional excipients and stabilizers. Additional active agents can vary widely in formulation methods and excipients, and this information is available for example, in Remington's Pharmaceutical Sciences (Arthur Osol, Editor).

Pharmaceutical compositions containing the antibodies described herein, or effective fragments thereof, may be formulated in combination with any suitable pharmaceutical vehicle, excipient or carrier that would commonly be used in this art, such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art.

Polyclonal antibody compositions can also be utilized. Preferably, the polyclonal antibody composition comprises purified antibodies, and does not comprise components of human serum, and therefore has the advantage of being free from contaminants or pathogens originating from a human donor. In certain embodiments, the polyclonal antibody composition comprises antibodies having specific binding for the same epitope of endotoxin. In other embodiments, the polyclonal antibody composition comprises antibodies having specific binding for more than one epitope of endotoxin. In an additional embodiment, the polyclonal antibody preparation Pentaglobin® can be utilized alone or in combination with additional endotoxin neutralizing agents.

The antibody compositions described herein are not limited to any particular source, and can be purified from antiserum, generated by recombinant methods, grown in hybridomas, CHO cells, plants, fungi, algae and so forth without limitation. The antibody compositions can also comprise antibody constructs such as humanized or chimerized antibodies, or fusion proteins. For example, E5 can be utilized in humanized form to facilitate clearance of bound endotoxin through the reticulendothelial system.

Endotoxin neutralizing agents, such as anti-endotoxin antibodies or LPS binding proteins, can also be utilized in the preparation of adsorbents, including immunoadsorbents, for use in plasmapheresis. Immunoadsorbents generally comprise an anti-endotoxin antibody or fragment thereof associated with a substrate (e.g., a sorbent) suitable for use in a plasmapheresis apparatus. Preferably, the anti-endotoxin antibody is selected from an antibody having specific binding for endotoxin, such as the core invariant glycolipid J5, and more preferably, an antibody such as HA-1A, E5, humanized or chimerized E5, or fragments or conjugates thereof, or recombinant versions thereof. In additional embodiments, the anti-endotoxin antibody is a VH4-34 antibody, such as mAb 216, which can be humanized or chimerized or fragments or conjugates thereof.

XI. Modes of Administration

The endotoxin neutralizing agents can be administered to the patient by a variety of different means and will vary depending upon the intended application. As one skilled in the art would recognize, administration of the therapeutic compositions can be carried out in various fashions, and more typically by parenteral injection into body cavity or vessel, e.g., intraperitoneal, intravenous, intralymphatic, intratumoral, intramuscular, interstitial, intraarterial, subcutaneous, intralesional, intraocular, intrasynovial, intraarticular. However, other methods of administration can be utilized for particular purposes, for example, via topical administration, including, but not limited to, dermal, ocular and rectal; transdermal, via passive or active means, e.g., using a patch, a carrier, or iontophoresis; transmucosal, e.g., sublingual, buccal, rectal, vaginal, or transurethral; oral, e.g., gastric or duodenal; via inhalation, e.g., pulmonary or nasal inhalation, using e.g., a nebulizer.

The endotoxin neutralizing agent can be administered as a bolus or over a period of time in a controlled delivery fashion, for example, as a continuous infusion (e.g., at a constant rate of delivery or at a varying rate) or in a pulsatile fashion. Continuous delivery can be provided for a period of time of minutes, hours or days, as needed to control the patient's endotoxemia.

The endotoxin neutralizing agent can be administered using a controlled delivery means, such as an infusion pump or an implanted device, or via diffusion from a matrix parenterally injected into a body cavity or site of endotoxin production. For example, a controlled delivery matrix containing endotoxin neutralizing agent, e.g., polylactide co-glycolide microspheres comprising endotoxin neutralizing agent, can be deposited subdurally or intramuscularly, to provide a controlled release of active agent over time.

In a preferred embodiment, the endotoxin neutralizing agent is administered as a continuous infusion over time to provide an amount of endotoxin neutralizing agent in the patient's blood in excess of the amount of endotoxin present over that same time period. Such a continuous delivery can provide a means for controlling the patient's endotoxemia even when endotoxin is continuously being released into the patient's blood, for example, by release from a leaky bowel or active gram negative infection. In a preferred embodiment, the rate of delivery of endotoxin neutralizing agent is sufficient to provide an equilibrium endotoxin level below that resulting in endotoxemia in the patient, preferably less than the threshold concentration of endotoxin for treatment, e.g., 5 pg/ml or most preferably, below the threshold of detection. Preferably, the rate of delivery of endotoxin neutralizing agent to the patient is greater than the rate of appearance of endotoxin in the patient's blood.

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of organic chemistry, polymer chemistry, immunochemistry, biochemistry and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

Abbreviations:

| | |
|---|---|
| ARDS | Adult respiratory distress syndrome |
| BPI | Bactericidal permeability inducing protein |
| Ig | Immunoglobulin |
| ELISA | Enzyme linked immunosorbent assay |
| LPS | Lipopolysaccharides |
| MODS | Multiorgan dysfunction syndrome |
| PMBC | Peripheral blood mononuclear cells |
| PMNL | Polymorphonuclear leukocytes |
| SIRS | Systemic inflammatory response syndrome |

### EXAMPLE 1

### Endotoxemia in Patients with Peritonitis from Ruptured Viscus

A double-blind randomized placebo-controlled clinical trial in patients with endotoxemia in the context of peritonitis due to ruptured viscus will be performed as follows: Hospitalized patients with peritonitis due to a ruptured viscus (ruptured appendix, ruptured diverticulum, colonic perforation, etc., resulting in the seeding of the peritoneum with fecal bacteria) will be treated with appropriate antibiotics and surgical intervention. Patients also will be monitored periodically for development of endotoxemia with a rapidly performed clinical assay of blood, plasma, or serum, for endotoxin (approximately every 6-8 hours or upon development of fever). Patients who are found to have endotoxemia of 5 pg/mL or greater will be randomized to receive prophylactically, either an endotoxin neutralizing agent (e.g. a monoclonal antibody that binds and neutralizes the biologic activity of endotoxin) or placebo. Patients will be monitored for development of septic shock, complications of endotoxemia (such as cardiac failure, pulmonary edema, hepatic injury, and renal dysfunction) and mortality. Analysis of the incidence of all endpoints will be compared between the endotoxin neutralizing agent treated group and the placebo treated group by statistical methods.

### EXAMPLE 2

### Endotoxemia in Patients with Neutropenia

A double-blind randomized placebo-controlled clinical trial in patients with endotoxemia in the context of neutropenia (<500 neutrophils mm³ of blood) will be performed as follows: Hospitalized patients with neutropenia in the context of recent chemotherapy or radiation therapy, or bone marrow aplasia, dysplasia, or leukemia, will be treated with appropriate antibiotics, and monitored periodically (approximately every 6-8 hours or upon development of a fever spike) for the development of endotoxemia using a rapidly performed clinical laboratory test for endotoxin in blood, plasma, or serum. Patients who are found to have endotoxemia of 5 pg/mL or greater will be randomized to receive prophylactically, either an endotoxin neutralizing agent (e.g. a monoclonal antibody that binds and neutralizes the biologic activity of endotoxin) or placebo. Patients will be monitored for development of septic shock, complications of endotoxemia (such as cardiac failure, pulmonary edema, hepatic injury, and renal dysfunction) and mortality. Analysis of the incidence of all endpoints will be compared between the endotoxin neutralizing agent treated group and the placebo treated group by statistical methods.

### EXAMPLE 3

### Endotoxemia in Patients with Urosepsis

A double-blind randomized placebo-controlled clinical trial in patients with endotoxemia in the context of urosepsis will be performed as follows: Hospitalized patients with urosepsis with gram negative organisms identified in a gram stain or microbiologic culture of urine will be treated with appropriate antibiotics, and monitored periodically (approximately every 6-8 hours or upon development of a fever spike) for the development of endotoxemia using a rapidly performed clinical laboratory test for endotoxin in blood, plasma, or serum. Patients who are found to have endotoxemia of 5 pg/mL or greater will be randomized to receive prophylactically, either an endotoxin neutralizing agent (e.g. a monoclonal antibody that binds and neutralizes the biologic activity of endotoxin) or placebo. Patients will be monitored for development of septic shock, complications of endotoxemia (such as cardiac failure, pulmonary edema, hepatic injury, and renal dysfunction) and mortality. Analysis of the incidence of all endpoints will be compared between the endotoxin neutralizing agent treated group and the placebo treated group by statistical methods.

### EXAMPLE 4

### Endotoxemia in Patients with Severe Liver Injury

A double-blind randomized placebo-controlled clinical trial in patients with endotoxemia in the context of severe liver injury will be performed as follows: Hospitalized patients with severe liver injury will be treated with appropriate antibiotics, and monitored periodically (approximately every 6-8 hours or upon development of a fever spike) for the development of endotoxemia using a rapidly performed clinical laboratory test for endotoxin in blood, plasma, or serum. Patients who are found to have endotoxemia of 5 pg/mL or greater will be randomized to receive prophylactically, either an endotoxin neutralizing agent (e.g. a monoclonal antibody that binds and neutralizes the biologic activity of endotoxin) or placebo. Patients will be monitored for development of septic shock, complications of endotoxemia (such as cardiac failure, pulmonary edema, worsening of hepatic injury, and renal dysfunction) and mortality. Analysis of the incidence of all endpoints will be compared between the endotoxin neutralizing agent treated group and the placebo treated group by statistical methods.

### EXAMPLE 5

### Endotoxemia in Patients with Severe Pancreatitis

A double-blind randomized placebo-controlled clinical trial in patients with endotoxemia in the context of severe pancreatitis will be performed as follows: Hospitalized patients with severe pancreatitis will be treated with appropriate antibiotics, and monitored periodically (approximately every 6-8 hours or upon development of a fever spike) for the development of endotoxemia using a rapidly performed clinical laboratory test for endotoxin in blood, plasma, or serum. Patients who are found to have endotoxemia of 5 pg/mL or greater will be randomized to receive prophylactically, either an endotoxin neutralizing agent (e.g. a monoclonal antibody that binds and neutralizes the biologic activity of endotoxin) or placebo. Patients will be monitored for development of septic shock, complications of endotoxemia (such as cardiac failure, pulmonary edema, hepatic injury, and renal dysfunction) and mortality. Analysis of the incidence of all endpoints will be compared between the endotoxin neutralizing agent treated group and the placebo treated group by statistical methods.

### EXAMPLE 6

### Endotoxemia in Patients with Leaky Bowel Syndrome

A double-blind randomized placebo-controlled clinical trial in patients with endotoxemia in the context of leaky bowel syndrome (i.e. abnormal passage of endotoxin from the fecal contents in the bowel lumen through the bowel mucosa into the blood in the context of severe systemic illness, as documented by an elevated level of calcitonin precursors in the blood) and fever will be performed as follows: Hospitalized patients with leaky bowel syndrome and fever will be treated with appropriate antibiotics, and monitored periodically (approximately every 6-8 hours or upon development of a fever spike) for the development of endotoxemia using a rapidly performed clinical laboratory test for endotoxin in blood, plasma, or serum. Patients who are found to have endotoxemia of 5 pg/mL or greater will be randomized to receive prophylactically, either an endotoxin neutralizing agent (e.g. a monoclonal antibody that binds and neutralizes the biologic activity of endotoxin) or placebo. Patients will be monitored for development of septic shock, complications of endotoxemia (such as cardiac failure, pulmonary edema, hepatic injury, and renal dysfunction) and mortality. Analysis of the incidence of all endpoints will be compared between the endotoxin neutralizing agent treated group and the placebo treated group by statistical methods.

### EXAMPLE 7

### Endotoxemia in Patients with Meningococcemia

A double-blind randomized placebo-controlled clinical trial in patients with endotoxemia in the context of meningococcemia (diagnosed by the presence of the characteristic rash and fever) will be performed as follows: Hospitalized patients with meningococcemia will be treated with appropriate antibiotics, and monitored periodically (approximately every 6-8 hours or upon development of a fever spike) for the development of endotoxemia using a rapidly performed clinical laboratory test for endotoxin in blood, plasma, or serum. Patients who are found to have endotoxemia of 5 pg/mL or greater will be randomized to receive prophylactically, either an endotoxin neutralizing agent (e.g. a monoclonal antibody that binds and neutralizes the biologic activity of endotoxin) or placebo. Patients will be monitored for development of septic shock, complications of endotoxemia (such as cardiac failure, pulmonary edema, hepatic injury, and renal dysfunction) and mortality. Analysis of the incidence of all endpoints will be compared between the endotoxin neutralizing agent treated group and the placebo treated group by statistical methods.

### EXAMPLE 8

### Endotoxemia in Patients with Elevated Levels of Calcitonin Precursors

A double-blind randomized placebo-controlled clinical trial in patients with endotoxemia in the context of elevated levels of calcitonin precursors in the blood and fever will be performed as follows: Hospitalized patients in the ICU with elevated levels of calcitonin precursors will be treated with appropriate antibiotics, and monitored periodically (approximately every 6-8 hours or upon development of a fever spike) for the development of endotoxemia using a rapidly performed clinical laboratory test for endotoxin in blood, plasma, or serum. Patients who are found to have endotoxemia of 5 pg/mL or greater will be randomized to receive prophylactically, either an endotoxin neutralizing agent (e.g. a monoclonal antibody that binds and neutralizes the biologic activity of endotoxin) or placebo. Patients will be monitored for development of septic shock, complications of endotoxemia (such as cardiac failure, pulmonary edema, hepatic injury, and renal dysfunction) and mortality. Analysis of the incidence of all endpoints will be compared between the endotoxin neutralizing agent treated group and the placebo treated group by statistical methods.

1. A method for selecting a patient suffering from endotoxemia for treatment with an endotoxin neutralizing therapy comprising:
(a) determining the level of endotoxin in the patient's blood; and
(b) comparing the endotoxin level in the patient's blood to a predetermined threshold endotoxin level to determine if the patient has elevated endotoxin levels.

2. The method of claim 1, further comprising treating patients identified as having elevated levels of endotoxin with an endotoxin neutralizing therapy selected from administering a pharmaceutical composition comprising a therapeutically effective amount of an endotoxin neutralizing agent to the patient, or performing plasmapheresis on the patient's blood using a plasmapheresis system comprising an endotoxin binding agent for removing endotoxin from the patient's blood, or combinations thereof.

3. The method of claim 1, wherein the threshold endotoxin level is 5-20 pg/mL.

4. The method of claim 3, wherein the threshold endotoxin level is 5 pg/ml.

5. The method of claim 1, wherein the elevated level of endotoxin is from about 5 pg/mL to at least about 100 pg/mL.

6. The method of claim 5, wherein the elevated level of endotoxin is from about 5 pg/mL to at least about 60 pg/mL.

7. The method of claim 1, wherein the patient is a human.

8. (canceled)

9. The method of claim 8, wherein the endotoxin neutralizing agent is an antibody having binding for endotoxin, LPS binding protein, or bactericidal permeability increasing protein.

10. The method of claim 9, wherein the antibody is a monoclonal or polyclonal antibody.

11. The method of claim 10, wherein the monoclonal antibody is selected from HA-1A, mAb 216, or E5, or a fragment, a fusion protein, a chimera, recombinant versions thereof, or combinations thereof.

12. The method of claim 10, wherein the monoclonal antibody is an IgM or an IgG, or a fragment, a fusion protein, a chimera, recombinant, or combinations thereof.

13. The method of claim 12, wherein the monoclonal antibody is an IgG₁ or an IgG₃.

14. The method of claim 1, wherein the endotoxemia is associated with gram negative bacteremia, gram positive bacteremia or fungemia.

15. The method of claim 1, wherein the endotoxemia is present without documentable bacteremia or fungemia.

16. The method of claim 14, wherein the endotoxemia is associated with infection with meningococcus.

17. The method of claim 14, wherein the endotoxemia is associated with a biowarfare agent.

18. The method of claim 17, wherein the biowarfare agent is Yersinia pestis, Franciella tularensis, Shigella sp., Salmonella sp., or other gram negative bacteria.

19. The method of claim 1, wherein the endotoxemia is associated with liver disease, pancreatitis, neutropenia or other immune suppression, or bowel edema/leaky gut due to severe systemic illness such as infection, trauma, ischemia, chemotherapy, radiation therapy, post-surgical state, or multiorgan dysfunction syndrome.

20. The method of claim 19, wherein the multiorgan dysfunction syndrome is selected from liver, lung, renal or cardiac dysfunction or failure.

21. The method of claim 2, further comprising administering an additional active agent to the patient.

22. The method of claim 21, wherein the additional active agent is selected from an antibiotic, a TLR-4 receptor antagonist, a cytokine inhibitor, an anti-inflammatory agent, or an anticoagulant, or combinations thereof.

23. The method of claim 22, wherein the TLR-4 receptor antagonist is E5564, B531 or TAK-242.

24. The method of claim 22, wherein the anti-inflammatory agent is a nonsteroidal anti-inflammatory agent selected from a salicylic acid derivative, an aryl propionic acid, a heteroaryl acetic acid, an indene acetic acid, a selective COX-2 inhibitor, an alkanone, an oxicam, or an anthranilic acid.

25. The method of claim 21, wherein the anticoagulant is an activated protein C, or heparin.

26. The method of claim 22, wherein the anti-inflammatory agent is an anti-inflammatory steroid.

27. The method of claim 26, wherein the anti-inflammatory steroid is selected from prednisone, prednisolone, methylprednisolone, triamcinolone or dexamethasone.

28. The method of claim 22, wherein the cytokine inhibitor is an IL-6 inhibitor, an IL-1 inhibitor, or a TNF inhibitor.

29. A method for treating a patient suffering from endotoxemia comprising:
(a) determining the level of endotoxin in the patient's blood;
(b) comparing the endotoxin level in the patient's blood to a predetermined threshold endotoxin level to determine if the patient has elevated endotoxin levels; and
(c) treating patients identified as having elevated levels of endotoxin with an endotoxin neutralizing therapy.

30. The method of claim 29, wherein the endotoxin neutralizing therapy is selected from administering a pharmaceutical composition comprising a therapeutically effective amount of an endotoxin neutralizing agent to the patient, or performing plasmapheresis on the patient's blood using a plasmapheresis system comprising an endotoxin binding agent for removing endotoxin from the patient's blood, or combinations thereof.

31. The method of claim 30, further comprising administering an additional active agent.

32. The method of claim 31, wherein the additional active agent is selected from a TLR-4 receptor antagonist, a cytokine inhibitor, a nonsteroidal anti-inflammatory agent, a steroidal anti-inflammatory agent, or an anticoagulant, or combinations thereof.

33. The method of claim 29, wherein the threshold endotoxin level is 5-20 pg/mL.

34. The method of claim 33, wherein the threshold endotoxin level is 5 pg/ml.

35. The method of claim 29, wherein the elevated level of endotoxin is from about 5 pg/mL to at least about 100 pg/mL.

36. The method of claim 35, wherein the elevated level of endotoxin is from about 5 pg/mL to at least about 60 pg/mL.

37. The method of claim 29, wherein the patient is a human.

38. The method of claim 30, wherein the endotoxin neutralizing agent is an antibody having binding for endotoxin, LPS binding protein, bactericidal permeability increasing protein, or TLR-4 receptor antagonist.

39. The method of claim 38, wherein the antibody is a monoclonal or polyclonal antibody.

40. The method of claim 39, wherein the monoclonal antibody is HA-1A, mAb 216, or E5, or a fragment, a fusion protein, a chimera, recombinant versions thereof, or combinations thereof.

41. The method of claim 40, wherein the monoclonal antibody is an IgM or an IgG, or a fragment, a fusion protein, a chimera, recombinant versions thereof, or combinations thereof.

42. The method of claim 41, wherein the monoclonal antibody is an IgG₁ or an IgG₃

43. The method of claim 29, wherein the endotoxemia is associated with gram negative bacteremia, gram positive bacteremia or fungemia.

44. The method of claim 29, wherein the endotoxemia is present without documentable bacteremia or fungemia.

45. The method of claim 43, wherein the endotoxemia is associated with infection with meningococcus.

46. The method of claim 29, wherein the endotoxemia is associated with a biowarfare agent.

47. The method of claim 46, wherein the biowarfare agent is Yersinia pestis, Franciella tularensis, Shigella sp., Salmonella sp., or other gram negative bacteria.

48. The method of claim 29, wherein the endotoxemia is associated with neutropenia or other immune suppression, liver disease, pancreatitis, bowel edema/leaky gut due to severe systemic illness such as infection, trauma, ischemia, chemotherapy, radiation therapy, post-surgical state, or multiorgan dysfunction syndrome (MODS).

49. The method of claim 48, wherein the multiorgan dysfunction syndrome is due to liver, renal, cardiac or lung dysfunction or failure.

50. The method of claim 29, wherein the endotoxemia is associated with peritonitis, neutropenia, urosepsis, severe liver injury, severe pancreatitis, leaky bowel syndrome, or meningococcemia.

51. A method for preventing septic shock, SIRS, MODS or mortality in a patient comprising:
(a) determining the level of endotoxin in the patient's blood;
(b) comparing the endotoxin level in the patient's blood to a predetermined threshold endotoxin level to determine if the patient has elevated endotoxin levels; and
(c) treating patients identified as having elevated levels of endotoxin with an endotoxin neutralizing therapy.

52. A method for reducing patient mortality, comprising:
(a) determining the level of endotoxin in a patient's blood;
(b) comparing the endotoxin level in the patient's blood to a predetermined threshold endotoxin level to determine if the patient has elevated endotoxin levels; and
(c) treating patients identified as having elevated levels of endotoxin with an endotoxin neutralizing therapy.

53. A method for reducing hospital and/or intensive care unit duration comprising:
(a) determining the level of endotoxin in a patient's blood;
(b) comparing the endotoxin level in the patient's blood to a predetermined threshold endotoxin level to determine if the patient has elevated endotoxin levels; and
(c) treating patients identified as having elevated levels of endotoxin with an endotoxin neutralizing therapy.

54. A method for reducing the incidence of morbidities in a patient, comprising:
(a) determining the level of endotoxin in the patient's blood;
(b) comparing the endotoxin level in the patient's blood to a predetermined threshold endotoxin level to determine if the patient has elevated endotoxin levels; and
(c) treating patients identified as having elevated levels of endotoxin with an endotoxin neutralizing therapy.

55. The method of claim 54, wherein the morbidities are selected from ARDS, SIRS, hepatic failure, renal failure, cardiac failure and multiorgan dysfunction syndrome (MODS).

56. A method for monitoring the therapeutic efficacy of a treatment for endotoxemia in a patient in need thereof, comprising:
(a) determining the level of endotoxin in the patient's blood;
(b) performing a treatment for endotoxemia;
(c) determining the level of endotoxin in the blood of a patient at a time after the treatment for endotoxemia;
(d) comparing the endotoxin level in the patient's blood to a predetermined threshold endotoxin level or the level of endotoxin prior to treatment to determine whether the endotoxin level in the patient's blood has decreased due to the treatment for endotoxemia or whether the endotoxin level remains elevated.

57. The method of claim 56, further comprising providing additional treatment for endotoxemia to patients identified as having elevated levels of endotoxin.

58. The method of claim 57, wherein the treatment for endotoxemia comprises plasmapheresis or the administration of a pharmaceutical composition comprising a therapeutically effective amount of an endotoxin neutralizing agent, or a combination thereof.

59. The method of claim 58, wherein the treatment for endotoxemia further comprises the administration of an additional active agent selected from an antibiotic, a TLR-4 receptor antagonist, a cytokine inhibitor, an anti-inflammatory agent, or an anticoagulant, or combinations thereof.

60. A method for monitoring the therapeutic efficacy of a treatment for endotoxemia in a patient in need thereof, comprising:
(a) determining the amounts of endotoxin and endotoxin neutralizing agent in the patient's blood; and
(b) comparing the amount of endotoxin neutralizing agent to the amount of endotoxin in the patient's blood to determine if the patient has sufficient endotoxin neutralizing agent to functionally neutralize the endotoxin.

61. The method of claim 60, further comprising
(c) administering an endotoxin neutralizing agent to patients identified as having an insufficient amount of endotoxin neutralizing agent to functionally neutralize the endotoxin in the patient's blood or performing plasmapheresis on the patient's blood using a plasmapheresis system comprising an endotoxin binding agent for removing endotoxin from the patient's blood, or combinations thereof.

62. The method of claim 61, wherein the endotoxin neutralizing agent administered in step (c) is the same or different from the endotoxin neutralizing agent in the patient's blood.

63. The method of claim 60, wherein the endotoxin neutralizing agent is selected from a LPS-binding protein, anti-endotoxin antibody, or bactericidal permeability inducing protein.

64. The method of claim 60, wherein the anti-endotoxin antibody is an endogenous anti-endotoxin antibody or an exogenous anti-endotoxin antibody.

65. The method of claim 64, wherein the exogenous anti-endotoxin antibody is selected from HA-1A, E5, Mab 216, recombinant versions thereof, fragments, fusion proteins, chimeras, or combinations thereof.

66. A method for monitoring the therapeutic efficacy of a treatment for endotoxemia in a patient in need thereof, comprising:
(a) determining the amount of endotoxin neutralizing agent in the patient's blood; and
(b) comparing the amount of endotoxin neutralizing agent in the patient's blood to the amount of endotoxin neutralizing agent that would be sufficient to functionally neutralize an endotoxin concentration of 5-1000 pg/ml in the patient's blood to determine if the patient has sufficient endotoxin neutralizing agent in their blood to functionally neutralize an endotoxin concentration of 5-1000 pg/ml.

67. The method of claim 66, further comprising
(c) administering an endotoxin neutralizing agent to patients identified as having an amount of endotoxin neutralizing agent in their blood that is not sufficient to functionally neutralize an endotoxin concentration of 5-1000 pg/ml.

68. A method for treating a patient suffering from a condition characterized by elevated levels of blood endotoxin, comprising:
(a) determining the level of endotoxin in the patient's blood;
(b) comparing the endotoxin level in the patient's blood to a predetermined threshold endotoxin level in the patient's blood to determine if the patient has elevated endotoxin levels; and
(c) treating patients identified as having elevated levels of endotoxin with an endotoxin neutralizing therapy,
said method providing a reduced risk of sepsis, septic shock, SIRS, MODS or mortality, and having improved safety relative to a method for administering an endotoxin neutralizing agent in the absence of elevated levels of plasma endotoxin.

69. A method for preventing septic shock, SIRS, MODS or mortality in a patient comprising:
(a) determining the level of calcitonin precursors in the patient's blood;
(b) comparing the level of calcitonin precursors in the patient's blood to a predetermined threshold level of calcitonin precursors in the patient's blood to determine if the patient has elevated calcitonin precursor levels; and if elevated, then measuring endotoxin levels in the blood of patients having elevated calcitonin precursors; and
(c) administering an endotoxin neutralizing agent to patients identified as having elevated levels of calcitonin precursors and endotoxin.

70. A method for reducing the risk of septic shock, SIRS, MODS or mortality in a patient suffering from pancreatitis or a condition characterized by leaky bowel syndrome or failure of gut barrier function, comprising:
(a) determining the level of calcitonin precursors in the patient's blood;
(b) comparing the level of calcitonin precursors in the patient's blood to a predetermined threshold calcitonin precursors in the patient's blood to determine if the patient has elevated calcitonin precursor levels; and if elevated, then measuring endotoxin levels in the blood of patients having elevated calcitonin precursors; and
(c) administering an endotoxin neutralizing agent to patients identified as having elevated levels of calcitonin precursors and endotoxin.

71. A method for treating a patient at risk for developing septic shock, SIRS, MODS or mortality, comprising:
(a) determining the level of endotoxin in the patient's blood;
(b) comparing the endotoxin level in the patient's blood to a predetermined threshold endotoxin level in the patient's blood to determine if the patient has elevated endotoxin levels; and
(c) administering a polyclonal antibody composition having binding specificity for at least one endotoxin epitope to patients identified as having elevated levels of endotoxin.

72. The method of claim 71, wherein the polyclonal antibody composition has binding specificity for more than one endotoxin epitope.

73. A pharmaceutical composition for treating a patient suffering from endotoxemia, comprising a therapeutically effective amount of at least one endotoxin neutralizing agent and a pharmaceutically acceptable carrier.

74. The composition of claim 73, wherein the composition comprises at least two endotoxin neutralizing agents.

75. The composition of claim 73, wherein the endotoxin neutralizing agent is an antibody, LPS binding protein, bactericidal permeability increasing protein, or combinations thereof.

76. The composition of claim 75, wherein the antibody is a monoclonal antibody selected from HA-1A, mAb 216, or E5, or a fragment, a fusion protein, a chimera, or recombinant versions thereof, or combinations thereof.

77. The composition of claim 75, wherein the antibody is an IgM or an IgG, or a fragment, a fusion protein, a chimera, recombinant versions thereof, or combinations thereof.

78. The composition of claim 76, wherein the monoclonal antibody is an IgG₁ or an IgG₃

79. The composition of claim 73, comprising at least two of the following endotoxin neutralizing agents: an antibody, a LPS binding protein, a bactericidal permeability increasing protein, a TLR4 receptor antagonist, or combinations thereof.

80. The composition of claim 73, further comprising an additional active agent.

81. The composition of claim 80, wherein the additional active agent is a TLR-4 receptor antagonist.

82. A composition for treating a patient suffering from endotoxemia, comprising a polyclonal antibody composition having binding specificity for at least one endotoxin epitope.

83. The composition of claim 82, wherein the polyclonal antibody composition has binding specificity for more than one endotoxin epitope.

84. A kit for selecting a patient suffering from endotoxemia for treatment with an endotoxin neutralizing agent comprising:
(a) means for determining the level of endotoxin in the patient's blood; and
(b) instructions for determining whether the patient has elevated levels of endotoxin in their blood; and optionally,
(c) at least one dose of a pharmaceutical composition comprising a therapeutically effective amount of an endotoxin neutralizing agent.

85. A kit for treating a patient suffering from endotoxemia comprising:
(a) means for determining the level of endotoxin in the patient's blood;
(b) instructions for determining whether the patient has elevated levels of endotoxin in their blood; and optionally
(c) at least one dose of a pharmaceutical composition comprising a therapeutically effective amount of an endotoxin neutralizing agent.

86. A kit for monitoring the therapeutic efficacy of a treatment for endotoxemia in a patient in need thereof, comprising:
(a) means for determining the level of endotoxin in the patient's blood before and after performing a treatment for endotoxemia;
(b) instructions for determining whether the patient has elevated levels of endotoxin in their blood; and optionally,
(c) at least one dose of a pharmaceutical composition comprising a therapeutically effective amount of an endotoxin neutralizing agent.

87. A kit for monitoring the therapeutic efficacy of a treatment for endotoxemia in a patient in need thereof, comprising:
(a) means for determining the amounts of endotoxin and endotoxin neutralizing agent in the patient's blood; and
(b) instructions for determining whether the patient has elevated levels of endotoxin in their blood and whether the patient has a sufficient amount of endotoxin neutralizing agent to functionally neutralize the amount of endotoxin in their blood or an amount of endotoxin of between about 5 and at least about 1000 pg/ml.

88. A kit for preventing septic shock, SIRS, MODS or mortality in a patient comprising:
(a) means for determining the level of calcitonin precursors and endotoxin levels in the patient's blood;
(b) instructions for determining whether the patient has elevated calcitonin precursors and endotoxin levels; and optionally
(c) at least one dose of a pharmaceutical composition comprising a therapeutically effective amount of an endotoxin neutralizing agent.

89. A kit for reducing the risk of septic shock, SIRS, MODS or mortality in a patient suffering from pancreatitis or a condition characterized by leaky bowel syndrome or failure of gut barrier function, comprising:
(a) means for determining the level of calcitonin precursors and endotoxin in the patient's blood;
(b) instructions for determining if the patient has elevated calcitonin precursors and endotoxin levels; and optionally
(c) at least one dose of a pharmaceutical composition comprising a therapeutically effective amount of an endotoxin neutralizing agent.

90. (canceled)

91. A method for controlling endotoxemia in a patient comprising:
(a) determining the level of endotoxin in the patient's blood;
(b) comparing the endotoxin level in the patient's blood to a predetermined threshold endotoxin level to determine if the patient has elevated endotoxin levels; and
(c) treating patients identified as having elevated levels of endotoxin with an endotoxin neutralizing therapy selected from administrating an endotoxin neutralizing agent to the patient, or treating the blood of the patient using plasmapheresis to reduce the level of endotoxin in the patient's blood, or combinations thereof.

92. The method of claim 91, wherein the level of endotoxin in the patient's blood is reduced below 100 pg/ml, more preferably below about 5-20 pg/ml and most preferably below 5 pg/ml.

93. The method of claim 92, wherein the level of endotoxin in the patient's blood is reduced below the threshold of detection.

94. The method of claim 2, wherein the endotoxin neutralizing agent is administered as a bolus or continuously over time.

95. The method of claim 91, wherein the endotoxin level in the patient is maintained at or below 5 pg/ml, and most preferably below the threshold of detection.
